(19) United States
(12) Patent Application Publication
(10) Pub. No.: US 2024/0350574 A1
(43) Pub. Date: Oct. 24, 2024

(54) PHENOLIC PHYTOCHEMICALS FOR BONE METASTASIS OF CANCER

(71) Applicant: NDSU RESEARCH FOUNDATION, Fargo, ND (US)

(72) Inventors: KALPANA S. KATTI, Fargo, ND (US); DINESH RAMANATH KATTI, Fargo, ND (US); Kalidas Shetty, Fargo, ND (US); Dipayan Sarkar, FARGO, ND (US); Preetham Ravi, FARGO, ND (US)

(21) Appl. No.: 18/640,277

(22) Filed: Apr. 19, 2024

Related U.S. Application Data
(60) Provisional application No. 63/497,335, filed on Apr. 20, 2023.

(51) Int. Cl. A61K 36/53; A61K 36/41; A61K 36/45; A61K 47/02; A61P 35/04

(52) U.S. Cl. CPC A61K 36/53; A61K 36/41; A61K 36/45; A61K 47/02; A61P 35/04

(57) ABSTRACT

Compositions comprising phenolic phytochemicals of oregano, cranberry, or Rhodiola are provided. Compositions comprising a polymer, clay, and the phenolic phytochemicals are also provided. Methods of using the phenolic phytochemical compositions to treat a subject with bone metastasis are also provided, particularly methods of treating bone metastasis from breast or prostate cancer.

### CROSS REFERENCE TO RELATED APPLICATIONS

This application claims priority under 35 U.S.C. § 119 to provisional patent application U.S. Ser. No. 63/497,335, filed Apr. 20, 2023. The provisional patent application is herein incorporated by reference in its entirety, including without limitation, the specification, claims, and abstract, as well as any figures, tables, appendices, or drawings thereof.

### STATEMENT REGARDING FEDERALLY SPONSORED RESEARCH OR DEVELOPMENT

This invention was made with government support under Grant No. 1946202 awarded by the National Science Foundation and Grant No. U54GM128729 awarded by the National Institutes of Health. The government has certain rights in the invention.

### TECHNICAL FIELD

The present disclosure relates generally to compositions and methods of using phenolic phytochemicals for the treatment of cancer, including cancer metastasis.

### BACKGROUND

Over 90% of cancer-associated deaths are attributed to metastasis, yet the mechanisms of metastasis remain poorly understood. Prostate cancer and breast cancer are hormonally regulated and have a strong propensity to metastasize to bone, at which point only palliative care is possible. Clinically, bone metastases are commonly observed in prostate cancer patients, with skeletal tumors observed in 10% of patients diagnosed with prostate cancer and 80% of patients with late-stage prostate cancer. Breast cancer metastasizes to the bone resulting in death of 70% of the patients within five years due to various bone-related complications such as hypercalcemia, bone fractures, cancer cachexia, and spinal cord compression. Similarly, for prostate cancer a 66.7% reduction in a five-year survival rate is observed. Further, bone metastasis causes significantly reduced quality of life due to bone pain and frequent fractures. There is currently no cure for bone metastasis of cancer. Furthermore, many drugs attempted have detrimental effects on bone.

Thus, there is a need in the art for new therapeutics that are more effective at treating advanced stage cancers and cancer metastasis and that have less side-effects to healthy tissue.

### SUMMARY

Methods of treating a subject having bone metastasis are provided. In some embodiments, the method comprises administering a composition comprising a phenolic phytochemical of oregano, cranberry, or Rhodiola to the subject. In some embodiments, the bone metastasis is from prostate cancer or breast cancer, including triple-negative breast cancer.

Compositions comprising phenolic phytochemicals of oregano, cranberry, or Rhodiola are also provided. In some embodiments, the composition comprises a polymer, a clay, and a phenolic phytochemical of oregano, cranberry, or Rhodiola. In some embodiments, the polymer and clay form a scaffold, and wherein the phenolic phytochemical coats or is impregnated in the scaffold or delivered to the scaffold via fluid flow in a bioreactor. In some embodiments, the composition is a nutraceutical composition.

Also provided are methods for screening a candidate compound for treatment of bone metastasis. In some embodiments, the method comprises contacting the candidate compound with a bone mimetic scaffold comprising a polymer, a clay, bone cells, and cancer cells.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows a schematic illustrating the process of creating the scaffolds and seeding cancer cells onto the scaffolds.

FIG. 2 is a bubble chart showing the binding affinities (kcal/mol) of Rhodiola compounds against breast cancer proteins in a grouped manner.

FIG. 3 is a bubble chart showing the binding affinities (kcal/mol) of Oregano compounds against breast cancer proteins.

FIGS. 4A-4C are bubble charts showing the binding affinities (kcal/mol) of Cranberry compounds against breast cancer proteins. FIGS. 5A and 5B are bar graphs comparing the cytotoxicity of Origanum vulgare (O.V.) on 2D and 3D cultures of breast cancer in two cell lines, MM231 (FIG. 5A) and MCF-7 (FIG. 5B). The concentrations of 0, 100, 200, 400, and 800 ppm were used. Alamar blue assay was used to evaluate cell viability. The asterisk symbol (*) indicates a statistically significant difference between drug-treated and non-treated samples. *p<0.05 **p<0.01, ***p<0.001.

FIGS. 6A and 6B are bar graphs comparing the cytotoxicity of Vaccinium macrocarpon (V.M.) on 2D and 3D cultures of breast cancer in two cell lines, MM231 (FIG. 6A) and MCF-7 (FIG. 6B). The concentrations of 0, 100, 200, 400, and 800 ppm were used. Alamar blue assay was used to evaluate cell viability. The asterisk symbol (*) indicates a statistically significant difference between drug-treated and non-treated samples. *p<0.05 **p<0.01, ***p<0.0001.

FIGS. 7A and 7B are bar graphs showing the effects of Vaccinium macrocarpon (7A) and Origanum vulgare (7B) on bone (33-day hMSC), after 24 hours. The concentration-dosages used 100, 200, 400, and 800 ppm. The asterisk symbol (*) indicates a statistically significant difference between drug-treated and non-treated samples. *p<0.05 **p<0.01 ***p<0.0001.

FIGS. 8A-8D show the cytotoxic effects of O.V. and V.M. were observed in 3D BM culture of breast cancer cells (MCF-7 and MM231) by live-dead staining (8A). 8B: Bar plots representing the percentages of apoptotic cells for 3D BM cultures of breast cancer cells MM231. 8C: Bar plots representing the percentages of apoptotic cells for 3D BM cultures of breast cancer cells MCF-7. The asterisk symbol (*) indicates significant difference in apoptotic percentage between drug treated sample and non-treated samples, in 2D and 3D culture. *p<0.05 **p<0.01 ***p<0.001. 8D is a representative fluorescent channel dot plot showing an analysis of 2D and 3D sequential cultures of breast cancer cells (MM 231 and MCF-7) showing double staining of Annexin V and Propidium Iodide. The cells were treated for 12 hours with their respective IC50 concentrations. The cells were double stained with Annexin V (x-axis) and Propidium Iodide (y-axis).

FIGS. 9A and 9B show pro-and anti-apoptotic markers p53 and bcl-2 analyzed using RT-PCR. 9A: Analysis of p53 and bcl-2 expression in 2D cultures of MCF-7 and BM culture of MCF-7 breast cancer cells. 9B: Analysis of p53 and bcl-2 expression in 2D cultures of MM231 and BM cultures of MM231. As seen, there is significant difference in relative expression for p53 and bcl-2 expression, between treated and untreated cultures of breast cancer. Cultures were treated with O.V. and V.M., the significance is indicated by *p<0.05, **p<0.01 ***p<0.001.

FIGS. 10A-10D show that apoptotic biomarkers experienced no significant changes when a healthy bone was treated with O.V. and V.M. 10A and 10B: Expressions of p53 and bcl-2 are represented by bone compared to the 3D culture of breast cancer cells (MM231 and MCF-7) after V.M. treatment. 10C and 10D: Expression of p53 and bcl-2 is represented by bone compared to 3D BM culture of breast cancer cells (MM231 and MCF-7) after O.V. treatment. Significance (*) indicates the significance of relative expression between treated and non-treated cultures. *p<0.05, **p<0.01 ***p<0.001.

FIGS. 11A-11C show live cell imaging of bone metastatic (BM) breast cancer cells (MM231 and MCF-7). 11A and 11B: ROS assay (green staining) and MtMP assay (red staining) were performed for treated and untreated samples. 11C: O.V. and V.M. treatment activates caspase-9 and caspase-3 in bone metastatic breast cancer (MM231 and MCF-7). Caspase-3 and caspase-9 expressions were measured after 24 hours of treatment. To indicate the significant difference in expression of caspase-9 between non-treated cultures and treated cultures of breast cancer cells (MM231 & MCF-7), *p<0.05, **p<0.01 ***p<0.001.

FIG. 12 is an image showing the tumor morphology of breast cancer cells on 2D and 3D cultures.

FIGS. 13A-13C show the cytotoxic effects of R. crenulata observed in 2D cultures of breast cancer cells (MM-231 and MCF-7) and 3D B.M. culture of breast cancer cells in (13A) MM-231 and (13B) MCF-7 cells, as well as prostate cancer cell (PC3 and MDA-Pca2b) (13C). The concentration dosages of 100, 200, 400, and 800 ppm of R. crenulata are used. Alamar blue assay was used to evaluate the cell viability. The asterisk symbol (*, **, ***) indicates a statistically significant difference between drug-treated and non-treated samples. *p<0.05 **p<0.01 ***p<0.001.

FIGS. 14A and 14B show the effects of R. crenulata on bone (33-day hMSC) (14A) and bone-met (CM) cultures of prostate cancer cells (14B). The concentration dosages used 100, 200, 400, and 800 ppm of R. crenulata.

FIGS. 15A-15C show (15A) The cytotoxic effects of R. crenulata observed in 2D cultures of breast cancer cells (MCF-7 and MM231) and 3D sequential culture of breast cancer cells (MCF-7 and MM231) by flow cytometric analysis. (15B) Bar plots represent apoptotic cell percentages for 2D and 3D B.M. cultures of breast cancer cells MCF-7. (15C) Bar plots represent apoptotic cell percentages for 2D and 3D B.M. cultures of breast cancer cells MM 231. The asterisk symbol (*) indicates significant difference in apoptotic percentage between drug-treated sample and non-treated samples, in 2D and 3D culture. *p<0.05 **p<0.01 ***p<0.0001.

FIGS. 16A-16D show pro-and anti-apoptotic markers p53 and bcl-2 analyzed using RT-PCR. (16A) Analysis of p53 expression in 2D and 3D B.M. cultures of MCF-7 breast cancer cells. (16B) Analysis of Bcl-2 expression in 2D and 3D B.M. culture of MCF-7 breast cancer cells. (16C) Analysis of p53 expression in 2D and 3D B.M. cultures of MM-231 breast cancer cells. (16D) Analysis of Bcl-2 expression in 2D and 3D B.M. cultures of MM-231 breast cancer cells. As seen, there is a significant difference in relative expression for p53, and bcl-2 expression between 2D and Bone-metastatic (B.M.) treated cultures of breast cancer, indicated by *p<0.05, **p<0.01 ***p<0.001

FIGS. 17A-17B show that apoptotic biomarkers experienced no significant changes when healthy bone was treated with R. crenulata. (17A) Expression of bcl-2 is represented by 33-day hMSCs (bone) compared to the 3D culture of breast cancer cells (MM 231 and MCF-7). (17B) Expression of p53 is represented by 33-day hMSCs (bone) compared to 3D B.M. culture of breast cancer cells (MM 231 and MCF-7). Significance (*) indicates the significance of relative expression between treated and non-treated cultures. *p<0.05, **p<0.01 ***p<0.001.

FIGS. 18A-18B show that R. crenulata activates caspase-9 and caspase-3 in bone metastatic breast cancer (MM 231 and MCF-7). (18A) Caspase-9 expression was measured after 24 hours of treatment. (18B) Caspase-3 expression was measured after 24 hours of treatment. To indicate the significant difference in expression of caspase-9 between non-treated cultures and treated cultures of breast cancer cells (MM 231 & MCF-7), *p<0.05, **p<0.01 ***p<0.001.

FIGS. 19A-19C show live/dead staining of patient derived breast cancer cell lines 24 hours after treatment with 800 ppm of Rhodiola, oregano, and cranberry. Live cells are stained green and dead cells are stained red. 19A: NT023 cells; 19B: NT015 cells; 19C: NT046 cells.

FIGS. 20A-20C show immunofluorescent staining of cleaved-caspase 3 (red) in patient derived breast cancer cells lines 12 hours after treatment with 800 ppm of Rhodiola, oregano, and cranberry. 20A: NT023 cells; 20B: NT015 cells; 20C: NT046 cells.

FIGS. 21A-21F show cell viability of prostate cancer cell lines PC3 and Pca2b following treatment with Rhodiola (21A-B), cranberry (21C-D), and oregano (21E-F). *p<0.05, **p<0.01, ***p<0.001 indicate a significant difference between non-treated and treated with 100 ppm, 200 ppm, 400 ppm, and 800 ppm.

Various embodiments of the present invention will be described in detail with reference to the figures, wherein like reference numerals represent like parts throughout the several views of various embodiments. Reference to various embodiments does not limit the scope of the invention. Figures represented herein are not limitations to the various embodiments according to the invention and are presented for exemplary illustration of the invention.

### DETAILED DESCRIPTION

The present disclosure is not to be limited to that described herein. Mechanical, electrical, chemical, procedural, and/or other changes can be made without departing from the spirit and scope of the present disclosure. No features shown or described are essential to permit basic operation of the present disclosure unless otherwise indicated.

This disclosure relates to phenolic phytochemicals extracted from food and medicinal plant sources (Rhodiola, cranberry and oregano). In vitro studies demonstrate the effectiveness of phenolic phytochemicals in causing apoptosis of cancer cells at bone site without any detrimental influence on bone and normal cells. Bone mimetic nanoclay testbeds that enable the evaluation of compounds, including unique combinations of compounds from the phenolic extracts, on bone metastasized cancer cells are also provided.

It is to be understood that all terminology used herein is for the purpose of describing particular embodiments only and is not intended to be limiting in any manner or scope. For example, as used in this specification and the appended claims, the singular forms “a,” “an” and “the” can include plural referents unless the content clearly indicates otherwise. Thus, for example, reference to “a cell” includes a single cell, as well as two or more cells. Similarly, the word “or” is intended to include “and” unless the context clearly indicate otherwise. The word “or” means any one member of a particular list and also includes any combination of members of that list. Further, all units, prefixes, and symbols may be denoted in its SI accepted form.

Numeric ranges recited within the specification are inclusive of the numbers defining the range and include each integer within the defined range. Throughout this disclosure, various embodiments of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾. This applies regardless of the breadth of the range.

The term “about,” as used herein, refers to variation in the numerical quantity that can occur, for example, through typical measuring techniques and equipment, with respect to any quantifiable variable, including, but not limited to, concentration, dosage, mass, temperature, time, and volume. Further, given solid and liquid handling procedures used in the real world, there is certain inadvertent error and variation that is likely through differences in the manufacture, source, or purity of the ingredients used to make the compositions or carry out the methods and the like. The term “about” also encompasses these variations. Whether or not modified by the term “about,” the claims include equivalents to the quantities.

The term “substantially” refers to a great or significant extent. “Substantially” can thus refer to a plurality, majority, and/or a supermajority of said quantifiable variables, given proper context. The term “generally” encompasses both “about” and “substantially.”

As used herein, the term “biocompatible” refers to materials that interact with the body, cells, or tissues without an undesirable effect.

As used herein, the term “exemplary” refers to an example, an instance, or an illustration, and does not indicate a most preferred embodiment unless otherwise stated.

The term “configured” describes structure capable of performing a task or adopting a particular configuration. The term “configured” can be used interchangeably with other similar phrases, such as constructed, arranged, adapted, manufactured, and the like.

As used herein, the term “biodegradable” refers to materials which can be metabolized by the body, e.g., enzymatically, chemically, or otherwise degrade in vivo or in vitro.

As used herein, the term “controlled release” refers to control of the rate of release, quantity released, or combination thereof of a therapeutic agent. A controlled release can be continuous or discontinuous, linear or non-linear.

As used herein, the term “phytochemical” refers to plant-based functional foods or isolated plant compounds or compounds present in fruits, vegetables, herbs, and spices. Therefore, a phytochemical can be defined as a non-nutrient bioactive compound present in a plant-based diet.

As used herein, the term “polymer” refers to a molecular complex comprised of more than ten monomeric units and generally includes, but is not limited to, homopolymers, copolymers, such as for example, block, graft, random and alternating copolymers, terpolymers, and higher “x”mers, wherein “x” is between 4 and 100, and further including their analogs, derivatives, combinations, and blends thereof. Furthermore, unless otherwise specifically limited, the term “polymer” shall include all possible isomeric configurations of the molecule, including, but are not limited to isotactic, syndiotactic and random symmetries, and combinations thereof. Furthermore, unless otherwise specifically limited, the term “polymer” shall include all possible geometrical configurations of the molecule.

The term “subject” as used herein refers to any living being that would benefit from the compositions and methods described herein. For example, the subject may be an animal, including a human, avian, bovine, canine, equine, feline, hircine, lupine, murine, ovine, and porcine animal. Subjects may also be domesticated animals such as cats, dogs, rabbits, guinea pigs, ferrets, hamsters, mice, gerbils, horses, cows, goats, sheep, donkeys, pigs, and the like. In certain embodiments, the subject is a human.

As used herein, the term “sustained release” refers to the continual release of a therapeutic agent over a period of time.

As used herein, “therapeutic agent” refers to any compound or composition of matter which, when administered to an organism (human or nonhuman animal) induces a desired pharmacologic, immunogenic, and/or physiologic effect by local and/or systemic action. The term therefore encompasses those compounds or chemicals traditionally regarded as drugs, vaccines, and biopharmaceuticals including molecules such as proteins, peptides, hormones, nucleic acids, gene constructs and the like.

As used herein, the term “therapeutic effect” means any improvement in the condition of a subject, human or animal, treated according to the subject method, including obtaining a preventative or prophylactic effect, or any alleviation of the severity of signs and symptoms of a disease, disorder, injury, or other condition which can be detected by means of physical examination, laboratory or instrumental methods.

As used herein, the terms “treat” and “treating” refer to: (i) alleviating the severity of signs and symptoms of a disease, disorder, injury, or other condition; (ii) inhibiting the a disease, disorder, injury, or other condition; and/or (iii) preventing a disease, disorder, injury, or other condition from occurring or recurring in an animal or human that may be predisposed to the disease, disorder and/or other condition.

Unless defined otherwise, all technical and scientific terms used above have the same meaning as commonly understood by one of ordinary skill in the art to which embodiments of the present disclosure pertain.

So that the present disclosure may be more readily understood, certain terms are first defined. Unless defined otherwise, all technical and scientific terms used herein have the same meaning as commonly understood by one of ordinary skill in the art to which embodiments of the disclosure pertain. Many methods and materials similar, modified, or equivalent to those described herein can be used in the practice of the embodiments of the present disclosure without undue experimentation, the preferred materials and methods are described herein. In describing and claiming the embodiments of the present disclosure, the following terminology will be used in accordance with the definitions set out below.

### Phenolic Phytochemical Compositions

The present disclosure provides phenolic phytochemicals of oregano, cranberry, and/or Rhodiola. As used herein, Rhodiola refers to the root extract of Rhodiola crenulata, oregano refers to the aerial part extract (leaf and flower) of Oreganum vulgare, and cranberry refers to extract from the pomace of the fruit of Vaccinium macrocarpon. These phytochemicals belong to different categories in their structure, i.e., polyphenols, flavonoids, flavonols, terpenoids, carotenoids, etc. In certain embodiments, the composition comprises an extract of oregano, cranberry and/or Rhodiola. In certain embodiments, the disclosure provides phenolic phytochemicals of other origin.

In certain embodiments, the phenolic phytochemical of oregano is carvacrol, thymol, creosol, phytol, p-cymene, gamma-terpinene, 1-octacosanol, luteolin 7-O-glucoside, rosmarinic acid, luteolin-7-O-glucuronide, apigenin-7-O-glucuronide, linalyl acetate, cis-sabinene hydrate, 4-hydroxy-4-methyl-2-pentanone, caffeic acid, trans-sabinene hydrate, quercetin 3-O-rutinoside, n-heptanoic acid, nitro-L-arginine, eriodictyol, taxifolin, dihydrokaempferol, or any combination thereof.

In certain embodiments, the phenolic phytochemical of cranberry is ursolic acid, cyanidin, cyanidin 3-O-galactoside, cyanidin 3-O-arabinoside, cyanidin 3-O-glucoside, peonidin, peonidin 3-O-galactoside, peonidin 3-O-arabinoside, peonidin 3-O-glucoside, malvidin, malvidin-3-O-galactoside, malvidin-3-O-arabinoside, pelargonidin, pelargonidin 3-O-arabinoside, delphinidin, delphinidin-3-O-arabinoside, petunidin, petunidin-3-O-galactoside, quercetin, hyperin, avicularin, quercitrin, isoquercitrin, quercetin 3-xyloside, kaempferol, kaempferol-3-glucoside, myricetin, myricetin 3-alpha-L-arabinofuranoside, -epicatechin, (+)-catechin, epigallocatechin, epigallocatechin gallate, (−)-catechin gallate, gallocatechin gallate, procyanidin b2, procyanidin a2, benzoic acid, salicylic acid, m-hydroxybenzoic acid, p-hydroxybenzoic acid, 2,3-dihydroxybenzoic acid, 2,4-dihydroxybenzoic acid, 3,4-dihydroxybenzoic acid, p-hydroxyphenylacetic acid, vanillic acid, trans-cinnamic acid, o-hydroxycinnamic acid, p-coumaric acid, o-phthalic acid, ferulic acid, sinapic acid, chlorogenic acid, 5-O-caffeoylquinic acid, phloridzin, ellagic acid, cis-resveratrol, trans-resveratrol, secoisolariciresinol, oleanolic acid, beta-sitosterol, sitogluside, monotropein, ascorbic acid, lutein, niacin, pantothenic acid, thiamine, riboflavin, adermine, folic acid, beta-carotene, alpha-tocopherol, or any combination thereof.

In certain embodiments, the phenolic phytochemical of Rhodiola is gallic acid, 3-O-methylgallic acid, 4-(β-d-glucopyranosyloxy)-3,5-dimethoxybenzoic acid, protocatechuic acid, vanillic acid, vanillic acid 4-O-β-d-glucopyranoside, tyrosol, salidroside, 4-hydroxybenzoic acid, 4-(beta-d-glucosyloxy) benzoic acid, rhodiocyanoside a, sarmentosin, epigallocatechin gallate, (7r*,8r*)-3-methoxy-3′,4,7,9,9′-pentahydroxy-8,4′-oxyneolignan 4-xyloside, isolariciresinol 4′-O-beta-d-glucoside, dehydrodiconiferyl alcohol 4-O-beta-d-glucopyranoside, picein, icariside d2, creoside i, kenposide a, rhodioloside e, rhodiooctanoside, coniferoside, dihydroconiferin, triandrin, vimalin, pollenitin, clemastanin a, or any epigallocatechin combination thereof.

In certain embodiments, the phenolic phytochemical comprises epigallocatechin gallate, malvidin-3-O-galactoside, quercitrin, folic acid, procyanidin a2, sitogluside, or any combination thereof.

Phenolic phytochemicals of the present disclosure can be incorporated into compositions, including nutraceutical compositions. Phenolic phytochemical compositions can optionally include additional functional ingredients suitable for uses disclosed herein. The functional ingredients provide desired properties and functionalities to the compositions. For the purpose of this application, the term “functional ingredient” includes a material that when dispersed or dissolved in the composition, such as an aqueous solution, emulsion or suspension, provides a beneficial property in a particular use. Some particular examples of functional materials are discussed in more detail below, although the particular materials discussed are given by way of example only, and that a broad variety of other functional ingredients may be used.

In some embodiments, the compositions may include suspending agents, rheology modifiers, surfactants, and other pharmaceutically-acceptable carriers and materials that do not negatively interfere with the phenolic phytochemicals. As referred to herein carriers and materials that do not negatively interfere with phenolic phytochemicals include food grade additives and materials. For example, liquid preparations may contain conventional additives such as suspending agents, for example sorbitol, syrup, methyl cellulose, hydroxyethylcellulose, glucose syrup, gelatin hydrogenated edible fats; emulsifying agents, for example lecithin, sorbitan monooleate, polysorbate 80, or acacia; non-aqueous vehicles (which may include edible oils), for example almond oil, fractionated coconut oil, oily esters such as glycerine, propylene glycol, or ethyl alcohol; preservatives, for example methyl or propyl p-hydroxybenzoate or sorbic acid, and if desired conventional flavoring or coloring agents.

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the composition and not injurious to the subject. According to embodiments, the pharmaceutically-acceptable carriers are preferred to further be food grade. Some examples of materials which can serve as pharmaceutically-acceptable carriers and/or food grade carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

According to embodiments of the disclosure, the various additional functional ingredients may be provided in a composition in the amount from about 0 wt-% and about 50 wt-%, from about 0.01 wt-% and about 50 wt-%, from about 0.1 wt-% and about 50 wt-%, from about 1 wt-% and about 50 wt-%, from about 1 wt-% and about 30 wt-%, from about 1 wt-% and about 25 wt-%, or from about 1 wt-% and about 20 wt-%. In addition, without being limited according to the disclosure, all ranges recited are inclusive of the numbers defining the range and include each integer within the defined range.

Compositions of the present disclosure may be formulated according to methods known in the art. Proper formulation is dependent upon the route of administration chosen. Suitable routes of administration include, but are not limited to, oral, parenteral (e.g., intravenous, intraarterial, rectal, subcutaneous, intramuscular, intraorbital, intracapsular, intraspinal, intraperitoneal, or intrasternal), topical (transdermal, intranasal, ocular, buccal, and sublingual), intravesical, intrathecal, enteral, pulmonary, intralymphatic, intracavital, vaginal, transurethral, intradermal, aural, intramammary, orthotopic, intratracheal, intralesional, percutaneous, endoscopical, transmucosal, and intestinal administration.

The formulations may conveniently be presented in unit dosage form. Formulations may be in the form of liquids, solutions, suspensions, emulsions, elixirs, syrups, tablets, lozenges, granules, powders, capsules, cachets, pills, ampoules, suppositories, pessaries, ointments, gels, pastes, creams, sprays, mists, foams, lotions, oils, boluses, electuaries, or aerosols.

Formulations suitable for oral administration (e.g., by ingestion) may be presented as discrete units such as capsules, cachets or tablets, each containing a predetermined amount of the active compound; as a powder or granules; as a solution or suspension in an aqueous or non-aqueous liquid; or as an oil-in-water liquid emulsion or a water-in-oil liquid emulsion; as a bolus; as an electuary; or as a paste.

A tablet may be made by conventional means, e.g., compression or molding, optionally with one or more accessory ingredients. Compressed tablets may be prepared by compressing in a suitable machine the active compound in a free-flowing form such as a powder or granules, optionally mixed with one or more binders (e.g. povidone, gelatin, acacia, sorbitol, tragacanth, hydroxypropylmethyl cellulose); fillers or diluents (e.g. lactose, microcrystalline cellulose, calcium hydrogen phosphate); lubricants (e.g. magnesium stearate, talc, silica); disintegrants (e.g. sodium starch glycolate, cross-linked povidone, cross-linked sodium carboxymethyl cellulose); surface-active or dispersing or wetting agents (e.g., sodium lauryl sulfate); and preservatives (e.g., methyl p-hydroxybenzoate, propyl p-hydroxybenzoate, sorbic acid). Molded tablets may be made by molding in a suitable machine a mixture of the powdered compound moistened with an inert liquid diluent. The tablets may optionally be coated or scored and may be formulated so as to provide slow or controlled release of the active compound therein using, for example, hydroxypropylmethyl cellulose in varying proportions to provide the desired release profile. Tablets may optionally be provided with an enteric coating, to provide release in parts of the gut other than the stomach.

Formulations suitable for topical administration (e.g. transdermal, intranasal, ocular, buccal, and sublingual) may be formulated as an ointment, cream, suspension, lotion, powder, solution, past, gel, spray, aerosol, or oil. Alternatively, a formulation may comprise a patch or a dressing such as a bandage or adhesive plaster impregnated with active compounds and optionally one or more excipients or diluents.

Formulations suitable for topical administration in the mouth include losenges comprising the active compound in a flavored basis, usually sucrose and acacia or tragacanth; pastilles comprising the active compound in an inert basis such as gelatin and glycerin, or sucrose and acacia; and mouthwashes comprising the active compound in a suitable liquid carrier.

Formulations suitable for topical administration to the eye also include eye drops wherein the active compound is dissolved or suspended in a suitable carrier, especially an aqueous solvent for the active compound.

Formulations suitable for nasal administration, wherein the carrier is a solid, include a coarse powder having a particle size, for example, in the range of about 20 to about 500 microns which is administered in the manner in which snuff is taken, i.e. by rapid inhalation through the nasal passage from a container of the powder held close up to the nose. Suitable formulations wherein the carrier is a liquid for administration as, for example, nasal spray, nasal drops, or by aerosol administration by nebuliser, include aqueous or oily solutions of the active compound.

Formulations suitable for administration by inhalation include those presented as an aerosol spray from a pressurized pack, with the use of a suitable propellant, such as dichlorodifluoromethane, trichlorofluoromethane, dichloro-tetrafluoroethane, carbon dioxide, or other suitable gases.

Formulations suitable for topical administration via the skin include ointments, creams, and emulsions. When formulated in an ointment, the active compound may optionally be employed with either a paraffinic or a water-miscible ointment base. Alternatively, the active compounds may be formulated in a cream with an oil-in-water cream base. If desired, the aqueous phase of the cream base may include, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof. The topical formulations may desirably include a compound which enhances absorption or penetration of the active compound through the skin or other affected areas. Examples of such dermal penetration enhancers include dimethylsulfoxide and related analogues.

When formulated as a topical emulsion, the oily phase may optionally comprise merely an emulsifier (otherwise known as an emulgent), or it may comprise a mixture of at least one emulsifier with a fat or an oil or with both a fat and an oil. Preferably, a hydrophilic emulsifier is included together with a lipophilic emulsifier which acts as a stabiliser. It is also preferred to include both an oil and a fat. Together, the emulsifier(s) with or without stabiliser(s) make up the so-called emulsifying wax, and the wax together with the oil and/or fat make up the so-called emulsifying ointment base which forms the oily dispersed phase of the cream formulations.

Suitable emulgents and emulsion stabilizers include Tween 60, Span 80, cetostearyl alcohol, myristyl alcohol, glyceryl monostearate and sodium lauryl sulphate. The choice of suitable oils or fats for the formulation is based on achieving the desired cosmetic properties, since the solubility of the active compound in most oils likely to be used in pharmaceutical emulsion formulations may be very low. Thus, the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for rectal administration may be presented as a suppository with a suitable base comprising, for example, cocoa butter or a salicylate. Formulations suitable for vaginal administration may be presented as pessaries, tampons, creams, gels, pastes, foams or spray formulations containing in addition to the active compound, such carriers as are known in the art to be appropriate.

The novel compounds of the present disclosure may also be preferably formulated for parenteral administration, e.g., formulated for injection via intravenous, intraarterial, subcutaneous, rectal, subcutaneous, intramuscular, intraorbital, intracapsular, intraspinal, intraperitoneal, or intrasternal routes. The compounds of the disclosure for parenteral administration comprise an effective amount of the novel compounds in a pharmaceutically acceptable carrier. Dosage forms suitable for parenteral administration include solutions, suspensions, dispersions, emulsions or any other dosage form which can be administered parenterally. Techniques and compositions for making parenteral dosage forms are known in the art.

Formulations suitable for parenteral administration (e.g., by injection, including cutaneous, subcutaneous, intramuscular, intravenous and intradermal), include aqueous and non-aqueous isotonic, pyrogen-free, sterile injection solutions which may contain antioxidants, buffers, preservatives, stabilizers, bacteriostats, and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. Examples of suitable isotonic vehicles for use in such formulations include Sodium Chloride Injection, Ringer's Solution, or Lactated Ringer's Injection. Typically, the concentration of the active compound in the solution is from about 1 ng/ml to about 10 μg/ml, for example from about 10 ng/ml to about 1 μg/ml. The formulations may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules, and tablets. Formulations may be in the form of liposomes or other microparticulate systems which are designed to target the active compound to blood components or one or more organs.

### Scaffolds

The present disclosure provides scaffolds comprising a clay and a polymer. The scaffold material may also include additional ingredients. The scaffolds can be prepared in small blocks. These blocks are defined as three dimensional elements of a customizable shape and size. The blocks can be prepared in any desired size. In certain embodiments, the blocks have porosities with at least one dimension that is at least 200 micrometers, at least 400 micrometers, or at least 500 micrometers. In certain embodiments, the blocks have porosities with at least two dimensions that are at least 200 micrometers, at least 400 micrometers, or at least 500 micrometers. In certain embodiments, the blocks have porosities with three dimensions that are at least 200 micrometers, at least 400 micrometers, or at least 500 micrometers. In addition, the blocks may also have porosities in the tens of micrometer and nanometer range. These porosities are verified using scanning electron microscopy imaging or micro tomography x-ray scanning. The purpose of these porosities is to allow for fluid flow, cell growth and tissue regeneration and the pore to solid volume in the scaffold should exceed 80%. In certain embodiments, the blocks have at least one dimension that is less than 50 millimeters, less than 25 millimeters, or less than 15 millimeters.

In certain embodiments, the scaffolds and/or scaffold blocks have at least two dimensions that are between 0.1 millimeters and 50 millimeters, between 0.5 millimeter and 25 millimeters, or between 1 millimeter and 15 millimeters. In certain embodiments, the blocks have three dimensions that are less than less than 50 millimeters, less than 25 millimeters, or less than 15 millimeters.

The blocks can be prepared in any desired shape. For example, the blocks can be prepared with straight edges or rounded edges. The blocks can be in the shape of a sphere, a cube, or any polygon. In the case of a polygon, the polygon can have any of its sides straight or rounded. In embodiments of the composition including more than one scaffold block, the scaffold blocks can be interconnected through geometrical interlocking, stacking, adhesion, or in any other suitable manner. The scaffold blocks can be prepared in a desired shape through fabrication or by cutting. These blocks can be made of scaffold materials as described herein.

The scaffolds and scaffold blocks can also contain any number of optional ingredients added for certain desired properties and/or effects.

The scaffolds and scaffold blocks can be sterilized. The scaffolds can facilitate or assist the generation of bone tissue, provide hierarchical structure for tissue to regenerate on, and/or provide a vehicle for the delivery of various optional ingredients. The scaffolds can allow for the use of the patient's own cells (autologous treatment) for bone regeneration. The generation of bone tissue on, over, and/or around the scaffolds can be seen based on the formation of an extracellular matrix and by seeing calcium formation.

### Clay

The scaffolds include a clay. As used herein, the clay is sometimes referred to as a nanoclay due to properties of the clay that are measured on the nanoscale. Suitable clays for forming the scaffolds can include, but are not limited to, smectite group of clay minerals such as, bentonite, beidellite, hectorite, nontronite, saponite, LAPONITE®, and combinations thereof. Other silicate minerals such as Halloysite, Silica nanoparticles, or quartz nanoparticles are also suitable. Reference to different species of clays includes the various types of that species, e.g., bentonite encompasses sodium bentonite, calcium bentonite, and potassium bentonite. In certain embodiments, bentonite clay includes sodium bentonite, calcium bentonite, and potassium bentonite. In certain embodiments, montmorillonite clay includes sodium montmorillonite and calcium montmorillonite and montmorillonite with other cations. In embodiments employing more than one clay, the multiple clays can be in a mixture or as separate clays.

The clay can comprise between 0.5 wt. % to 99.5 wt. % of the scaffold or scaffold block. In certain embodiments, the clay can comprise between 1 wt. % to 80 wt. % of the scaffold or scaffold block. In certain embodiments, the clay can comprise between 5 wt. % to 75 wt. % of the scaffold or scaffold block. In certain embodiments, the scaffolds have between 1 wt. % and about 20 wt. % clay, between about 2 and 15 wt. %, or between about 5 wt. % and about 20 wt. %.

### Polymer

The scaffolds include a polymer. The compositions can include more than one polymer. In certain embodiments, the polymer is biocompatible. In certain embodiments, the polymer can be biodegradable and/or conductive.

Suitable polymers for use in the scaffolds include any polymeric material without limitation so long as it possesses the necessary biocompatible and/or biodegradable properties. In certain embodiments, polymers include those of natural and synthetic origins, and blends, combinations, or mixtures of the same, which can be formed into copolymers, terpolymers, or “x”mers.

Examples of natural polymers include, but are not limited to, proteins and polysaccharides, which can be used individually, in blends, combinations and/or mixtures, Natural polymers, include, but are not limited to, albumin, alginate, cellulose (which is inclusive of regenerated cellulose), chitin, chitosan, collagen, gelatin, heparin, and other naturally occurring polymers such as regenerated silk or polysaccharide, and/or blends, combinations, or mixtures of the same.

Examples of synthetic polymers include, but are not limited to, poly(amino acids), polyanhydrides, polyesters, poly(alpha-hydroxy acids), poly(lactones), poly(orthocarbonates), poly(orthoesters), poly(phosphoesters), or polyphosphazenes, which can be used individually, in blends, combinations and/or mixtures. Synthetic polymers, include, but are not limited to, polycaprolactone (PCL), poly(delta-valerolactone), poly(1,5-dioxepan-2-one), poly(epsilon-caprolactone), poly(ester urethane) (PEU), polygalactouronic acid, poly(gamma-butyrolactone), polyglycolic acid, poly(alpha-hydroxy acids), polyhydroxyalkanoate (PHA), polyhydroxybutyric acid, poly(3-hydroxybutyrate-co-3-hydroxyvalerate (PHBV), polylactic acid (PLA) (e.g., poly(DL-lactic acid) and poly(L-lactic acid)), copolymers of lactic acid-glycolic acid such as poly(lactic-co-glycolic acid) (PLGA), poly(lactic acid-co-caprolactone) (PLCL), poly(trimethylene carbonate), poly-8-valerolactone, or blends, combinations, and mixtures of the same.

Examples of conductive polymers include, but are not limited to, polyacrylonitrile, polyimide, and regenerated cellulose. In certain embodiments, the polymer, scaffold, and/or scaffold black can be coated with a conductive material. In certain embodiments, conductive polymers may be preferred for in situ sensor applications and for evaluation of degradation.

In certain embodiments, polymers include, but are not limited to, chitosan-polygalactouronic acid and polycaprolactone.

The polymer can comprise between 1 wt. % to 99.5 wt. % of the scaffold or scaffold block. In certain embodiments, the polymer can comprise between 25 wt. % to 95 wt. % of the scaffold or scaffold block. In certain embodiments, the polymer can comprise between 50 wt. % to 90 wt. % of the scaffold or scaffold block.

### Additional Ingredients

The scaffolds and scaffold blocks can optionally contain any number of additional ingredients added for certain desired properties and/or effects. The additional ingredients can be naturally occurring or synthetic, organic or inorganic. Suitable additional ingredients, include, but are not limited to amino acids, anesthetics, antibiotics, antiangiogenic agents, antibodies, anticoagulants, antineoplastic agents, antiviral agents, biomaterials, bone morphogenetic proteins, carbohydrates, cells, cytotoxic agents, drugs, electrolytes, growth factors, immunomodulators, inorganic materials, lipids, minerals (such as hydroxyapatite mineral (HAP)), oligonucleotides, osteoblasts, osteoclasts, osteo stem cells, polypeptides, progenitors, proteins, stem cells (adult and/or embryonic), therapeutic agents, tissues, tissue or cell aggregates, vasoactive agents, and combinations thereof. Preferred proteins include bone morphogenetic protein (BMP), particularly BMP-2 and BMP-7.

Increased backbone length of amino acids can increase molecular interaction between polymer, amino acid and clay allowing for significant improvement in the mechanical property. Some functional groups of the amino acids can increase the molecular interaction between polymer, amino acid and clay leading to significant improvement in the mechanical properties. Unnatural amino acids provide longer backbone chains and are thus candidates as modifiers. In certain embodiments, it is preferable to have an amino acid with a carbon backbone chain length of at least five carbon atoms. In certain embodiments, amino acids have a carbon chain length of between one and about ten. In certain embodiments, amino acids include but are not limited to, aminovaleric, amino caprylic, amino pimelic acids, aminobutyric acid, and combinations thereof.

In certain embodiments, the scaffold can comprise phenolic phytochemicals of oregano, cranberry, and/or Rhodiola. In some embodiments, the phenolic phytochemical coats the scaffold. In some embodiments, the phenolic phytochemical is impregnated in the scaffold.

The additional ingredients can be selected to impart particular functionalities or properties. For example, additional ingredients can be selected to affect and/or control the mechanical, biological and degradation properties of the scaffold. In certain embodiments, specific additional ingredients can be selected for the desired properties or effects and based on the patient. For example, in the case of a human patient, a human osteoblast can be used, whereas if the patient is a cow a bovine osteoblast can be used. Similarly, certain additional ingredients can be specifically tailored to the patient based on use of their own genetic and/or cellular materials, e.g., cell lines developed based on compatibility or directly from the patient's own genetic and/or cellular materials. In certain embodiments, the scaffolds and/or scaffold blocks can incorporate autologous treatments.

Any suitable amount of additional ingredients can be used in the scaffolds and scaffold blocks. The appropriate amount of an additional ingredient can be dictated by the patient's condition, age, size, general health, medical conditions, allergies, etc. Generally, the additional ingredients will be included in an amount of between 0.01 wt. % and 50 wt. % of the composition. The additional ingredients can be part of the scaffolds and/or scaffold blocks, impregnated within the scaffolds and/or scaffold blocks, coat the scaffolds or scaffold blocks, or any combination thereof. The additional ingredients can be attached to, coat, and/or modify the clay or polymer. In certain embodiments, when used to coat the scaffolds, the additional ingredients can be prepared in a solution and the scaffolds can be soaked in the solution.

### Methods of Preparing the Scaffolds and Scaffold Blocks

A freeze-drying method can be used to prepare the scaffolds. In such a method, the polymer is first dissolved in a solvent followed by slowly adding the clay. In a preferred embodiment, the clay can be modified by one or more of the additional ingredients as described above. Any suitable method for modifying the clay can be used. After adding the clay to the dissolved polymer, another solvent can be added to the resultant followed by controlled freeze drying.

Other suitable methods of preparing scaffolds includes 3D printing, electrospinning, spinning or other methods for polymer clay nanocomposite fiber preparation.

Any suitable solvent can be used to dissolve the polymer and the solvent can be selected based on the polymer to be dissolved. Similarly, any suitable solvent can be used for the freeze-drying step. Preferred solvents for the freeze-drying step include those having a melting point that is below that of the scaffold components. An exemplary alcohol useful for freeze drying is isopropyl alcohol.

An exemplary method for preparing scaffolds according to a preferred embodiment of the invention is as follows. For example, purposes, a polymer solution can be prepared and a clay can be added. Preferably, the mixture of clay and polymer can be centrifuged. In a preferred embodiment, the mixture of polymer and clay is allowed to dry to form a film before centrifuging. Next a polymer composite solution (solution of polymer and clay) can be cooled to freeze. A solvent, such as an alcohol, can be added during the freezing step. The solvent should be allowed to freeze into crystals. The composite with frozen solvent can then be immersed in an extraction solvent (for example, another alcohol, preferably ethanol) so that the solvent crystals can be extracted. Preferably the extraction solvent is replaced at regular intervals (e.g., daily, every 12 hours, etc.). Finally, the porous composite structure, now a scaffold, is removed and dried under laboratory conditions. This scaffold can now be cut and prepared in a desired shape. In another embodiment of the invention, the freezing steps can be performed in a mold of the desired shape and dimensions. Additional ingredients can be added prior to the freezing step or after the scaffold is removed. For example, if certain additional ingredients are desired to be part of the scaffold, they can be added prior to the freezing step. However, in other embodiments, it may be preferred to add the additional ingredients after the scaffold is prepared, e.g., when seeding a scaffold with cells.

Composite scaffolds containing in situ HAPclay at a desired concentration and PCL can be prepared as described in FIG. 3. Suitable concentrations of the clay can be as set forth above. Cylindrical shaped frozen samples of the composite solution can be carefully removed from polypropylene (PP) centrifuge tubes and further immersed in absolute ethanol (cooled to −20° C.) for solvent extraction. The cylindrical shaped porous samples, known as scaffolds, were removed and dried at room temperature.

Some of the optional ingredients can be prepared in a “biomimetic” manner in situ. For example, precursors to the desired optional ingredient can be added to the space between the clay sheets on the nanoscale so that an optional ingredient is formed between the sheets of clay. For example, amino acid molecules can be attached to the clay sheets to create HAP in a manner similar to the way HAP is naturally made in bones. This “biomimetic” in situ HAP-clay is morphologically and crystallographically identical to the natural HAP found in bones, while made in a synthetically engineered manner.

Individual scaffolds and/or scaffold blocks can be prepared and/or selected based on size and shape. They can be combined to fit a bone defect or injury. Suitable optional ingredients can be added to the scaffolds and/or scaffold blocks. The scaffolds and scaffold blocks can be added to a bone injury or defect through the appropriate medical treatment such as traditional surgery or minimally invasive surgery (e.g., arthroscopic or laparoscopic).

### Methods of Use/Treatment

Methods of treating a subject with cancer comprising administering phenolic phytochemical compositions are provided. Without being limited by theory, the phenolic phytochemicals induce apoptosis of bone metastatic cancer cells. In some embodiments, the phenolic phytochemicals are derived from Rhodiola, cranberry, or oregano.

In certain embodiments, the subject has bone metastasis. In certain embodiments, the bone metastasis is from prostate cancer or breast cancer. In certain embodiments, the bone metastasis is from triple-negative breast cancer. As used herein, “triple-negative breast cancer” refers to a type of breast cancer wherein the cancer cells lack receptors for estrogen, progesterone, and human epidermal growth factor (HER2).

Suitable routes of administration include, but are not limited to, oral, parenteral (e.g., intravenous, intraarterial, rectal, subcutaneous, intramuscular, intraorbital, intracapsular, intraspinal, intraperitoneal, or intrasternal), topical (transdermal, intranasal, ocular, buccal, and sublingual), intravesical, intrathecal, enteral, pulmonary, intralymphatic, intracavital, vaginal, transurethral, intradermal, aural, intramammary, orthotopic, intratracheal, intralesional, percutaneous, endoscopical, transmucosal, and intestinal administration. In certain embodiments, the composition is administered orally, intravenously, or intramuscularly.

The size of the dose of each therapy which is required for the therapeutic or prophylactic treatment of a particular disease state will necessarily be varied depending on the host treated, the route of administration and the severity of the illness being treated. Accordingly, the optimum dosage may be determined by the practitioner who is treating any particular patient and taking into consideration various factors known to modify the action of drugs including severity and type of disease, body weight, sex, diet, time and route of administration, other medications and other relevant clinical factors. It may also be necessary or desirable to reduce the doses of the components of the combination treatments in order to reduce toxicity. Therapeutically effective dosages may be determined by either in vitro or in vivo methods.

The dosages and schedules may vary according to the particular disease state and the overall condition of the patient. Dosages and schedules may also vary if, in addition to administration of a composition of the present disclosure, one or more additional chemotherapeutic agents is/are used. Scheduling can be determined by the practitioner who is treating any particular patient.

In an embodiment, the treatment may be performed by administration of the phenolic phytochemical composition to the subject in need thereof or by administration directly to the site of the tumor or other cancer cells. The treatment may be performed in conjunction with administration of a chemotherapeutic agent or additional treatment agent (e.g., as part of a treatment regimen), either simultaneously, in a single composition or in separate compositions, or sequentially, with the phenolic phytochemical compositions of the present disclosure. The treatment may be performed by administration of components in any order and in any combination. The treatment may also be performed using more than one chemotherapeutic agent, or other type of treatment. Further, the treatment may be performed by providing multiple administrations of the compositions. One skilled in the art will ascertain these variations in treatment regimens employing the novel compounds disclosed herein.

The phenolic phytochemical compositions of the disclosure can be used in combination with at least one or more chemotherapeutic agents. In some embodiments, the chemotherapeutic may comprise mitotic inhibitors, alkylating agents, anti-metabolites, intercalating antibiotics, growth factor inhibitors, cell cycle inhibitors, enzymes, topoisomerase inhibitors, biological response modifiers, anti-hormones, angiogenesis inhibitors, and anti-androgens. Non-limiting examples are chemotherapeutic agents, cytotoxic agents, and non-peptide small molecules such as Gleevec® (Imatinib Mesylate), Kyprolis® (carfilzomib), Velcade® (bortezomib), Casodex (bicalutamide), Iressa® (gefitinib), Venclexta™ (venetoclax) and Adriamycin™, (docorubicin) as well as a host of chemotherapeutic agents. Non-limiting examples of chemotherapeutic agents include alkylating agents such as thiotepa and cyclosphosphamide (Cytoxan™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, chlorocyclophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, Casodex™, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfomithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g. paclitaxel and docetaxel; retinoic acid; esperamicins; capecitabine; and pharmaceutically acceptable salts, acids or derivatives of any of the above.

Also included as suitable chemotherapeutic cell conditioners are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY 117018, onapristone, and toremifene (Fareston); and anti-androgens such as flutamide, nilutamide, bicalutamide, leuprolide, and goserelin; chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; camptothecin-11 (CPT-11); topoisomerase inhibitor RFS 2000; difluoromethylornithine (DMFO).

In certain embodiments, phenolic phytochemical compositions of the present disclosure may be administered in combination with radiation therapy. Techniques for administering radiation therapy are known in the art, and these techniques can be used in the method described herein. Radiation therapy may be administered before, during, and/or after treatment with compounds and compositions of the present disclosure.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### NUMBERED EMBODIMENTS

1. A method for treating a subject with bone metastasis, the method comprising: administering a composition comprising a phenolic phytochemical of oregano, cranberry, or Rhodiola and an excipient to the subject.
2. The method of embodiment 1, wherein the bone metastasis is from prostate cancer or breast cancer.
3. A method of inducing apoptosis of bone metastatic breast cancer cells or bone metastatic prostate cancer cells in a subject, the method comprising: administering a composition comprising a phenolic phytochemical of oregano, cranberry, or Rhodiola to the subject.
4. The method of any one of embodiments 1-3, wherein the composition comprises an extract of oregano, cranberry, Rhodiola, or a combination thereof.
5. The method of any one of embodiments 1-4, wherein the phenolic phytochemical of Rhodiola comprises Gallic acid, 3-O-methylgallic acid, 4-(D-D-Glucopyranosyloxy)-3,5-dimethoxybenzoic acid, protocatechuic acid, vanillic acid, vanillic acid 4-O-β-D-glucopyranoside, Tyrosol, Salidroside, 4-Hydroxybenzoic acid, 4-(beta-D-Glucosyloxy) benzoic acid, Rhodiocyanoside A, Sarmentosin, Epigallocatechin gallate, (7R*,8R*)-3-Methoxy-3′,4,7,9,9′-pentahydroxy-8,4′-oxyneolignan 4-xyloside, Isolariciresinol 4′-O-beta-D-glucoside, Dehydrodiconiferyl alcohol 4-O-beta-D-glucopyranoside, Picein, Icariside D2, Creoside I, Kenposide A, Rhodioloside E, Rhodiooctanoside, coniferoside, dihydroconiferin, triandrin, Vimalin, Pollenitin, Clemastanin A, or a combination thereof.
6. The method of any one of embodiments 1-5, wherein the phenolic phytochemical of oregano comprises Carvacrol, Thymol, Creosol, Phytol, P-Cymene, Gamma-Terpinene, 1-Octacosanol, Luteolin 7-O-glucoside, Rosmarinic acid, Luteolin-7-o-glucuronide, Apigenin-7-o-glucuronide, Linalyl acetate, cis-Sabinene hydrate, 4-Hydroxy-4-Methyl-2-Pentanone, Caffeic acid, Trans-Sabinene hydrate, Quercetin 3-O-rutinoside, n-Heptanoic acid, Nitro-L-arginine, Eriodictyol, Taxifolin, Dihydrokaempferol, or a combination thereof.
7. The method of any one of embodiments 1-6, wherein the phenolic phytochemical of cranberry comprises Ursolic acid, Cyanidin, Cyanidin 3-O-galactoside, Cyanidin 3-O-arabinoside, Cyanidin 3-O-glucoside, Peonidin, Peonidin 3-O-galactoside, Peonidin 3-O-arabinoside, Peonidin 3-O-glucoside, Malvidin, Malvidin-3-O-galactoside, Malvidin-3-O-arabinoside, Pelargonidin, Pelargonidin 3-O-arabinoside, Delphinidin, Delphinidin-3-O-arabinoside, Petunidin, Petunidin-3-O-galactoside, Quercetin, Hyperin, Avicularin, Quercitrin, Isoquercitrin, Quercetin 3-xyloside, Kaempferol, Kaempferol-3-glucoside, Myricetin, Myricetin 3-alpha-L-arabinofuranoside, -Epicatechin, (+)-Catechin, Epigallocatechin, Epigallocatechin gallate, (−)-Catechin gallate, Gallocatechin gallate, Procyanidin B2, Procyanidin A2, Benzoic acid, Salicylic acid, M-hydroxybenzoic acid, p-Hydroxybenzoic acid, 2,3-Dihydroxybenzoic acid, 2,4-Dihydroxybenzoic acid, 3,4-Dihydroxybenzoic acid, p-hydroxyphenylacetic acid, Vanillic acid, Trans-Cinnamic acid, o-Hydroxycinnamic acid, p-coumaric acid, o-phthalic acid, Ferulic acid, Sinapic acid, Chlorogenic acid, 5-O-Caffeoylquinic acid, Phloridzin, Ellagic acid, cis-Resveratrol, trans-resveratrol, Secoisolariciresinol, Oleanolic acid, Beta-Sitosterol, Sitogluside, Monotropein, Ascorbic acid, Lutein, Niacin, Pantothenic acid, Thiamine, Riboflavin, Adermine, Folic acid, beta-Carotene, alpha-Tocopherol, or a combination thereof.
8. The method of any one of embodiments 1-7, wherein the composition comprises epigallocatechin gallate, Malvidin-3-O-galactoside, Quercitrin, folic acid, procyanidin A2, Sitogluside, or a combination thereof.
9. The method of any one of embodiments 1-8, wherein the composition is administered orally.
10. The method of any one of embodiments 1-9, wherein the composition is not cytotoxic to healthy bone cells.
11. A composition comprising: a polymer, a clay, a phenolic phytochemical of oregano, cranberry, or Rhodiola, wherein the polymer and clay form a scaffold, and wherein the phenolic phytochemical coats or is impregnated in the scaffold.
12. The composition of embodiment 11, wherein the composition comprises an extract of oregano, cranberry, Rhodiola, or a combination thereof.
13. The composition of embodiment 11 or embodiment 12, wherein the phenolic phytochemical of Rhodiola comprises Gallic acid, 3-O-methylgallic acid, 4-(D-D-Glucopyranosyloxy)-3,5-dimethoxybenzoic acid, protocatechuic acid, vanillic acid, vanillic acid 4-O-β-D-glucopyranoside, Tyrosol, Salidroside, 4-Hydroxybenzoic acid, 4-(beta-D-Glucosyloxy) benzoic acid, Rhodiocyanoside A, Sarmentosin, Epigallocatechin gallate, (7R*,8R*)-3-Methoxy-3′,4,7,9,9′-pentahydroxy-8,4′-oxyneolignan 4-xyloside, Isolariciresinol 4′-O-beta-D-glucoside, Dehydrodiconiferyl alcohol 4-O-beta-D-glucopyranoside, Picein, Icariside D2, Creoside I, Kenposide A, Rhodioloside E, Rhodiooctanoside, coniferoside, dihydroconiferin, triandrin, Vimalin, Pollenitin, Clemastanin A, or a combination thereof.
14. The composition of any one of embodiments 11-13, wherein the phenolic phytochemical of oregano comprises Carvacrol, Thymol, Creosol, Phytol, P-Cymene, Gamma-Terpinene, 1-Octacosanol, Luteolin 7-O-glucoside, Rosmarinic acid, Luteolin-7-o-glucuronide, Apigenin-7-o-glucuronide, Linalyl acetate, cis-Sabinene hydrate, 4-Hydroxy-4-Methyl-2-Pentanone, Caffeic acid, Trans-Sabinene hydrate, Quercetin 3-O-rutinoside, n-Heptanoic acid, Nitro-L-arginine, Eriodictyol, Taxifolin, Dihydrokaempferol, or a combination thereof.
15. The composition of any one of embodiments 11-14, wherein the phenolic phytochemical of cranberry comprises Ursolic acid, Cyanidin, Cyanidin 3-O-galactoside, Cyanidin 3-O-arabinoside, Cyanidin 3-O-glucoside, Peonidin, Peonidin 3-O-galactoside, Peonidin 3-O-arabinoside, Peonidin 3-O-glucoside, Malvidin, Malvidin-3-O-galactoside, Malvidin-3-O-arabinoside, Pelargonidin, Pelargonidin 3-O-arabinoside, Delphinidin, Delphinidin-3-O-arabinoside, Petunidin, Petunidin-3-O-galactoside, Quercetin, Hyperin, Avicularin, Quercitrin, Isoquercitrin, Quercetin 3-xyloside, Kaempferol, Kaempferol-3-glucoside, Myricetin, Myricetin 3-alpha-L-arabinofuranoside, -Epicatechin, (+)-Catechin, Epigallocatechin, Epigallocatechin gallate, (−)-Catechin gallate, Gallocatechin gallate, Procyanidin B2, Procyanidin A2, Benzoic acid, Salicylic acid, M-hydroxybenzoic acid, p-Hydroxybenzoic acid, 2,3-Dihydroxybenzoic acid, 2,4-Dihydroxybenzoic acid, 3,4-Dihydroxybenzoic acid, p-hydroxyphenylacetic acid, Vanillic acid, Trans-Cinnamic acid, o-Hydroxycinnamic acid, p-coumaric acid, o-phthalic acid, Ferulic acid, Sinapic acid, Chlorogenic acid, 5-O-Caffeoylquinic acid, Phloridzin, Ellagic acid, cis-Resveratrol, trans-resveratrol, Secoisolariciresinol, Oleanolic acid, Beta-Sitosterol, Sitogluside, Monotropein, Ascorbic acid, Lutein, Niacin, Pantothenic acid, Thiamine, Riboflavin, Adermine, Folic acid, beta-Carotene, alpha-Tocopherol, or a combination thereof.
16. The composition of any one of embodiments 11-15, wherein the composition comprises epigallocatechin gallate, Malvidin-3-O-galactoside, Quercitrin, folic acid, procyanidin A2, Sitogluside, or a combination thereof.
17. The composition of any one of embodiments 11-16, wherein the clay is modified with hydroxyapatite.
18. The composition of any one of embodiments 11-17, wherein the polymer is biocompatible 19. The composition of any one of embodiments 11-18, wherein the polymer is a natural polymer, synthetic polymer, or blend, combination, or mixture thereof.
20. The composition of any one of embodiments 11-19, wherein the polymer comprises albumin, alginate, cellulose, chitin, chitosan, collagen, gelatin, heparin, regenerated silk polymer, polysaccharide, poly(amino acid), polyanhydride, polyester, poly(alpha-hydroxy acid), poly(lactone), poly(orthocarbonate), poly(orthoester), poly(phosphoester), polyphosphazenes, blend, mixture, combination thereof.
21. The composition of any one of embodiments 11-20, wherein the polymer comprises polyacrylonitrile, polycaprolactone, poly(delta-valerolactone), poly(1,5-dioxepan-2-one), poly(epsilon-aprolactone), poly(ester urethane), polygalactouronic acid, poly(gamma-butyrolactone), polyglycolic acid, poly(alpha-hydroxy acids), polyhydroxyalkanoate, polyhydroxybutyric acid, poly(3-hydroxybutyrate-co-3-hydroxyvalerate, polyimide, polylactic acid, poly(lactic-co-glycolic acid), poly(lactic acid-co-caprolactone), poly(trimethylene carbonate), poly-8-valerolactone, or blends, combinations, and mixtures of the same.
22. The composition of any one of embodiments 11-21, wherein the polymer comprises chitosan-polygalactouronic acid, polycaprolactone, or a blend, combination, or mixture thereof.
23. The composition of any one of embodiments 11-22, wherein the clay comprises a smectite.
24. The composition of any one of embodiments 11-23, wherein the clay comprises bentonite, beidellite, hectorite, montmorillonite, nontronite, saponite, or combinations thereof.
25. The composition of any one of embodiments 11-24, wherein the clay comprises sodium bentonite, calcium bentonite, potassium bentonite, sodium montmorillonite, calcium montmorillonite, or combinations thereof.
26. The composition of any one of embodiments 11-25, wherein the scaffold is formed into one or more scaffold blocks.
27. The composition of any one of embodiments 11-26, wherein the clay comprises between 0.5 wt. % to 99.5 wt. % of the scaffold or the scaffold block 28. The composition of any one of embodiments 11-27, wherein the polymer comprises between 0.1 wt. % to 99.5 wt. % of the scaffold or the scaffold block.
29. The composition of any one of embodiments 11-28, wherein the scaffold or the scaffold block further comprises an additional ingredient of an amino acid, anesthetic, antibiotic, antiangiogenic agent, antibody, anticoagulant, antineoplastic agent, antiviral agent, biomaterial, carbohydrate, cell, cytotoxic agent, drug, electrolyte, growth factor, immunomodulator, inorganic material, lipid, mineral, oligonucleotide, osteoblast, osteoclast, osteo stem cell, polypeptide, progenitor, protein, therapeutic agent, tissue, tissue or cell aggregate, vasoactive agent, and combinations thereof.
30. The composition of embodiment 29, wherein the additional ingredient is between 0.01 wt. % and 50 wt. % of the composition.
31. The composition of embodiment 29 or embodiment 30, wherein the additional ingredient is attached to, coats, or modifies the scaffold, scaffold block, clay, and/or polymer.
32. The composition of any one of embodiments 29-31, wherein the additional ingredient is impregnated in the scaffold or the scaffold block.
33. The composition of any one of embodiments 29-32, wherein one or more of the additional ingredients is released by a controlled release and/or sustained release.
34. The composition of any one of embodiments 29-33, wherein the additional ingredient comprises one or more of a human osteoblast, a non-human animal species osteoblast, an amino acid, a growth factor, a bone morphogenic protein, and/or an adult stem cell.
35. A method for treating a subject with bone metastasis, the method comprising: administering the composition of any one of embodiments 11-35 to a bone of the subject.
36. A nutraceutical composition comprising: a phenolic phytochemical of oregano, cranberry, or Rhodiola; and an excipient.
37. The nutraceutical composition of embodiment 36, wherein the composition comprises an extract of oregano, cranberry, Rhodiola, or a combination thereof.
38. The nutraceutical composition of embodiment 36 or embodiment 37, wherein the phenolic phytochemical of Rhodiola comprises Gallic acid, 3-O-methylgallic acid, 4-(D-D-Glucopyranosyloxy)-3,5-dimethoxybenzoic acid, protocatechuic acid, vanillic acid, vanillic acid 4-O-β-D-glucopyranoside, Tyrosol, Salidroside, 4-Hydroxybenzoic acid, 4-(beta-D-Glucosyloxy) benzoic acid, Rhodiocyanoside A, Sarmentosin, Epigallocatechin gallate, (7R*,8R*)-3-Methoxy-3′,4,7,9,9′-pentahydroxy-8,4′-oxyneolignan 4-xyloside, Isolariciresinol 4′-O-beta-D-glucoside, Dehydrodiconiferyl alcohol 4-O-beta-D-glucopyranoside, Picein, Icariside D2, Creoside I, Kenposide A, Rhodioloside E, Rhodiooctanoside, coniferoside, dihydroconiferin, triandrin, Vimalin, Pollenitin, Clemastanin A, or a combination thereof.
39. The nutraceutical composition of any one of embodiments 36-38, wherein the phenolic phytochemical of oregano comprises Carvacrol, Thymol, Creosol, Phytol, P-Cymene, Gamma-Terpinene, 1-Octacosanol, Luteolin 7-O-glucoside, Rosmarinic acid, Luteolin-7-o-glucuronide, Apigenin-7-o-glucuronide, Linalyl acetate, cis-Sabinene hydrate, 4-Hydroxy-4-Methyl-2-Pentanone, Caffeic acid, Trans-Sabinene hydrate, Quercetin 3-O-rutinoside, n-Heptanoic acid, Nitro-L-arginine, Eriodictyol, Taxifolin, Dihydrokaempferol, or a combination thereof.
40. The nutraceutical composition of any one of embodiments 36-39, wherein the phenolic phytochemical of cranberry comprises Ursolic acid, Cyanidin, Cyanidin 3-O-galactoside, Cyanidin 3-O-arabinoside, Cyanidin 3-O-glucoside, Peonidin, Peonidin 3-O-galactoside, Peonidin 3-O-arabinoside, Peonidin 3-O-glucoside, Malvidin, Malvidin-3-O-galactoside, Malvidin-3-O-arabinoside, Pelargonidin, Pelargonidin 3-O-arabinoside, Delphinidin, Delphinidin-3-O-arabinoside, Petunidin, Petunidin-3-O-galactoside, Quercetin, Hyperin, Avicularin, Quercitrin, Isoquercitrin, Quercetin 3-xyloside, Kaempferol, Kaempferol-3-glucoside, Myricetin, Myricetin 3-alpha-L-arabinofuranoside, -Epicatechin, (+)-Catechin, Epigallocatechin, Epigallocatechin gallate, (−)-Catechin gallate, Gallocatechin gallate, Procyanidin B2, Procyanidin A2, Benzoic acid, Salicylic acid, M-hydroxybenzoic acid, p-Hydroxybenzoic acid, 2,3-Dihydroxybenzoic acid, 2,4-Dihydroxybenzoic acid, 3,4-Dihydroxybenzoic acid, p-hydroxyphenylacetic acid, Vanillic acid, Trans-Cinnamic acid, o-Hydroxycinnamic acid, p-coumaric acid, o-phthalic acid, Ferulic acid, Sinapic acid, Chlorogenic acid, 5-O-Caffeoylquinic acid, Phloridzin, Ellagic acid, cis-Resveratrol, trans-resveratrol, Secoisolariciresinol, Oleanolic acid, Beta-Sitosterol, Sitogluside, Monotropein, Ascorbic acid, Lutein, Niacin, Pantothenic acid, Thiamine, Riboflavin, Adermine, Folic acid, beta-Carotene, alpha-Tocopherol, or a combination thereof.
41. The nutraceutical composition of any one of embodiments 36-40, wherein the composition comprises epigallocatechin gallate, Malvidin-3-O-galactoside, Quercitrin, folic acid, procyanidin A2, Sitogluside, or a combination thereof.
42. The nutraceutical composition of any one of embodiments 36-41, wherein the composition induces apoptosis of bone metastatic breast cancer cells or bone metastatic prostate cancer cells.
43. The nutraceutical composition of any one of embodiments 36-42, wherein the composition is not cytotoxic to bone cells.
44. A method for screening a candidate compound for treatment of bone metastasis, the method comprising: contacting the candidate compound with a bone mimetic scaffold comprising a polymer, a clay, bone cells, and cancer cells.
45. The method of embodiment 44, further comprising evaluating the cytotoxicity of the candidate compound to the cancer cells, and optionally evaluating the cytotoxicity of the candidate compound to the bone cells.
46. The method of embodiment 44 or embodiment 45, wherein the cancer cells are breast cancer cells or prostate cancer cells.
47. The method of any one of embodiments 44-46, wherein the clay is modified with hydroxyapatite.

### EXAMPLES

Embodiments of the present invention are further defined in the following nonlimiting Examples. It should be understood that these Examples, while indicating certain embodiments of the invention, are given by way of illustration only. From the above discussion and these Examples, one skilled in the art can ascertain the essential characteristics of this invention, and without departing from the spirit and scope thereof, can make various changes and modifications of the embodiments of the invention to adapt it to various usages and conditions. Thus, various modifications of the embodiments of the invention, in addition to those shown and described herein, will be apparent to those skilled in the art from the foregoing description. Such modifications are also intended to fall within the scope of the appended claims.

### Example 1

A study was conducted to investigate the cytotoxicity of Rhodiola crenulata on bone metastatic breast cancer derived from a unique 3D in vitro bone-mimetic scaffold and to uncover the mechanism of cell apoptosis in bone metastatic breast cancer.

### Materials and Methods

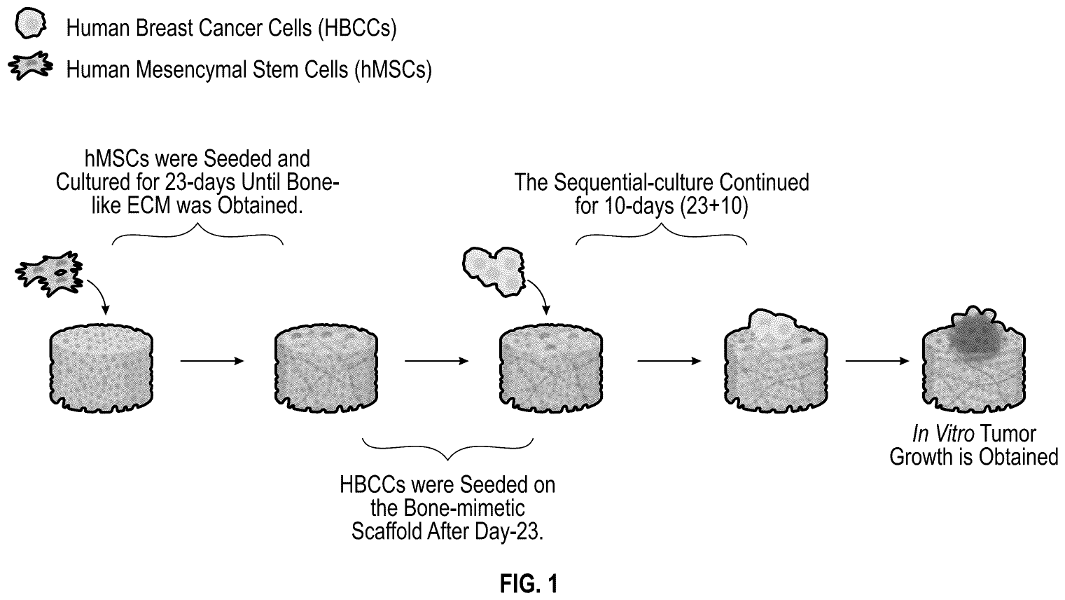

A unique 3D in vitro bone-mimetic scaffold, using the sequential culture of human mesenchymal stem cells (hMSCs) and human breast cancer cells, to accurately recapitulate bone metastatic breast cancer was developed. As shown in the steps of FIG. 1, the PCL/in situ HAPclay scaffolds had tailored mechanical properties, through clay modifications, and achieve bone-like tissue formation via vesicular delivery of osteogenically differentiated hMSCs 2, 3. Human breast cancer cells were then seeded on the scaffold after 23-days, cultured for 10-days 3, 4.

PCL/in situ HAPclay scaffolds were prepared by modifying Na-MMT clay with 5-aminovaleric acid to increase the d-spacing of the clay. Hydroxyapatite (HAP) was then biomineralized in the intercalated nano-clay galleries, generating in situ HAP clay. Finally, the PCL and 10% in situ HAPclay were dissolved in 1,4-dioxane, and employing the freeze-drying method, PCL/in situ HAP clay scaffolds were prepared. The dimensions of the scaffolds used for experiments was 12 mm in diameter and 3 mm in thickness.

A concentration of 1000 ppm of stock solution of Rhodiola crenulata was prepared by dissolving 0.01 g of Rhodiola crenulata (One World Products, 980106) in 100 mL of 10% ethanol solution. This targeted phenolic phytochemicals rich therapeutic solution was passed through a 0.22 μm filter for sterilization. The stock solution was further diluted in serial dilutions using serum-free DMEM media (ATCC).

MDA-MB 231 (MM 231) cells were cultured in 90% DMEM, 10% FBS, and 1% Penicillin-Streptomycin (P/S). MCF-7 cells were grown in 90% Eagle's Minimum Essential Medium (EMEM), 10% FBS, 0.01 mg/mL human recombinant insulin, and 1% P/S. All cell cultures were maintained at 37° C. and 5% CO2 in a humidified incubator. For 2D cultures, 1×105 breast cancer cells (MM 231/MCF-7) were cultured on tissue culture polystyrene (TCPS). For 3D bone-metastatic (BM) cultures, scaffolds were first sterilized under UV light for 45 min, followed by immersion in 70% ethanol for 24 hours. Further, scaffolds were washed in PBS twice and placed in 24-well plates containing culture medium and stored in humidified 5% CO2 incubator at 37° C. Scaffolds were seeded with 1×105 MSCs per scaffold and kept for 4 hours for cell adherence before adding culture medium. Further, cells were cultured for 23 days to obtain bone-like extracellular matrix (ECM) formation. Next, 1×105 breast cancer cells (MM 231/MCF-7) were seeded per scaffold on bone ECM formed on the scaffolds and cultured in breast cancer cell medium.

2D cultures of breast cancer cells (at day 10) and 3D cultures of breast cancer cells on bone ECM were the serum-starved for 24 hours and subsequently treated with different Rhodiola crenulata concentrations (0, 100, 200, 400, 800 PPM) for 24 hours. Cell viability of treated and untreated samples was determined using a Alamar blue cell viability reagent (Invitrogen), using the manufacturer's protocol. Half maximal inhibitory concentration (IC50) values for 2D and 3D cultures were calculated with Graph Pad Prism (v7.04) using nonlinear regression analysis.

Live/dead assay was performed on normal bone cultures, untreated and treated with 800 ppm concentration with R.C. Cells were stained with a live/Dead™ Cell Imaging Kit (Thermofisher Scientific, Germany) according to the manufacturer's protocol. Live/dead solution was prepared by mixing the Calcein AM solution and BOBO-3 iodide. Samples were washed with PBS twice. Next, treated and untreated samples were introduced with both live stain (Calcein AM) and dead stain (BOBO-3 iodide)). Prior to imaging, samples were incubated for 15 minutes at room temperature. Samples were imaged using JPK Nanowizard Bio-AFM confocal system.

After treating 2D cultures of breast cancer cells and 3D sequential cultures of MSCs with breast cancer cells for 12 hours with their respective IC50 concentrations, the cells were harvested and washed in cold PBS 3 times. Further, the cells were resuspended in cold Annexin Binding Buffer to a concentration of 1×10⁶ cells/mL, then labeled with Propidium Iodide (PI) and Fluorescein isothiocyanate (FITC)-conjugated Annexin V and analyzed using BD Accuri C6 Flow cytometer and software.

Prior to the cancer cells seeding step, hMSCs were cell cycle-arrested with 10 ug/mL of Mitomycin B. Cancer cells were grown for 10 days in both 2D and 3D cultures and treated with IC50 drug concentration. After 24 hours of treatment, total RNA was isolated from cell-seeded scaffolds and 2D cultures using Direct-zol RNA MiniPrep kit. Isolated RNA was reverse transcribed to synthesize cDNA using M-MLV reverse transcriptase (Promega), random primers, and thermal cycler (Applied Biosystems). Real-Time Polymerase Chain Reaction (RT-PCR) was performed using a thermal profile with a holding stage (2 min at 50° C., 10 min at 95° C.) and a cycling stage (40 cycles of 15 s at 95° C., and 1 min at 60° C.) on 7500 Fast Real-Time System (Applied Biosystems). The mRNA expression of p53, and Bcl-2 were evaluated in both 2D cultures of breast cancer cells (at day 10) and 3D cultures of breast cancer cells (at day (23+10)). All the mRNA expressions were normalized to the housekeeping gene glyceraldehyde-3-phosphate dehydrogenase (GAPDH). Target gene expressions were calculated using the comparative Ct method (2^{-ΔΔCt}).

Cells grown in 3D and 2D cultures were then fixed in 4% paraformaldehyde in PBS for 45 min, permeabilized with 0.2% TritonX-100 in PBS for 5 min, and blocked with 0.2% blocking buffer (0.2% FSG in PBS containing 0.02% Tween20) for 45 min. Further, the samples were incubated with primary antibodies overnight at 4° C. The actin cytoskeleton and nuclei of cells were stained with Rhodamine Phalloidin and DAPI, respectively. The samples were observed with JPK Nanowizard Bio-AFM confocal system.

For the reactive oxygen species (ROS) assay. Live MM231 and MCF-7 bone metastatic cultures were stained following the manufacturer's protocols. Briefly, samples were treated with R.C. for 12 hours and then washed with PBS. Next, DCFDA solution was diluted and added to samples in the dark for 45 minutes at 37° C. Lastly, all samples were rinsed with buffer. Imaging was performed using JPK nanowizard bio-AFM confocal system with FITC filter.

For mitochondrial membrane potential (MtMP) assay, Manufacturer's protocols were followed. MM231 and MCF-7 bone metastatic cultures were treated with R.C. for 12 hours. Next, samples were washed with PBS. 200 nM of TMRE labeling solution was added to all live samples. Samples were placed in CO2 incubator for 20 minutes. Live cell imaging was performed using JPK nanowizard bio-AFM confocal system, using the orange-red filter.

The data shown are calculated as mean±standard derivation (n=3). Using one-way ANOVA, the statistical significance or p-values among multiple comparisons were determined, followed by post hoc Tukey test. The differences between the two groups were determined to be statistically significant utilizing an unpaired Student's t-test, using GraphPad Prism v7.04. p<0.05 was considered statistically significant.

The 3D and 2D cultures were utilized in the following examples to evaluate the cytotoxicity of three phenolic compounds, Origanum vulgare, Vaccinium macrocarpon, and Rhodiola crenulata, The unique 3D in vitro bone-mimetic testbed was shown to be a viable and reliable screening tool for identifying new potential anti-cancer drugs and therapies.

### Example 2

The cytotoxicity of Rhodiola crenulata on bone metastatic breast cancer was evaluated using 3D in vitro nano-clay based scaffold models, testbeds for bone metastatic breast cancer drug screening applications, and compared the results with 2D cultured cells. Scaffolds and cultured cells were prepared as described in Example 1.

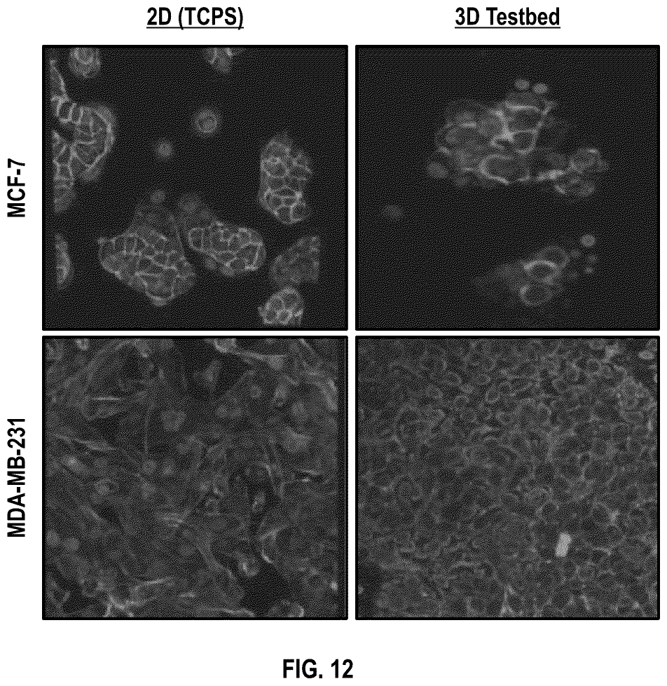

To investigate the morphology of the breast cancer cells on the 3D bone mimetic testbed, the actin cytoskeleton was stained with rhodamine phalloidin and compared to the results with breast cancer cells grown on 2D substrate. FIG. 12 shows the tumor morphology of 2D and 3D cultures that was determined by staining of actin cytoskeleton with rhodamine phalloidin, along DAPI for nuclei staining. As shown in FIG. 12, the breast cancer cells that were grown on 2D TCPS were flat and did not experience tumor formation. Additionally, it was found that MCF-7 cells formed clusters on 2D substrate. However, at the bone microenvironment, MCF-7 formed clustered-compact tumoroids, indicating strong cell-cell interactions. Thus, it was found that breast cancer cells grown on bone-mimetic 3D testbed form in-vivo like tumoroids.

Cell viability assay was used to evaluate the cytotoxic effects of R. crenulata on 2D and 3D cultures. Cells were treated with 100, 200, 400, and 800 ppm of R. crenulata for 24 hours and the dose-dependent cytotoxic effects were observed in MM 231 and MCF-7 cell cultures. Alamar blue assay was used to evaluate the cell viability. The IC50 was calculated from the dose-response study using curve-fitting. The asterisk symbol (*) indicates significant difference between drug treated sample and non-treated samples. *p<0.05 **p<0.01 ***p<0.001.

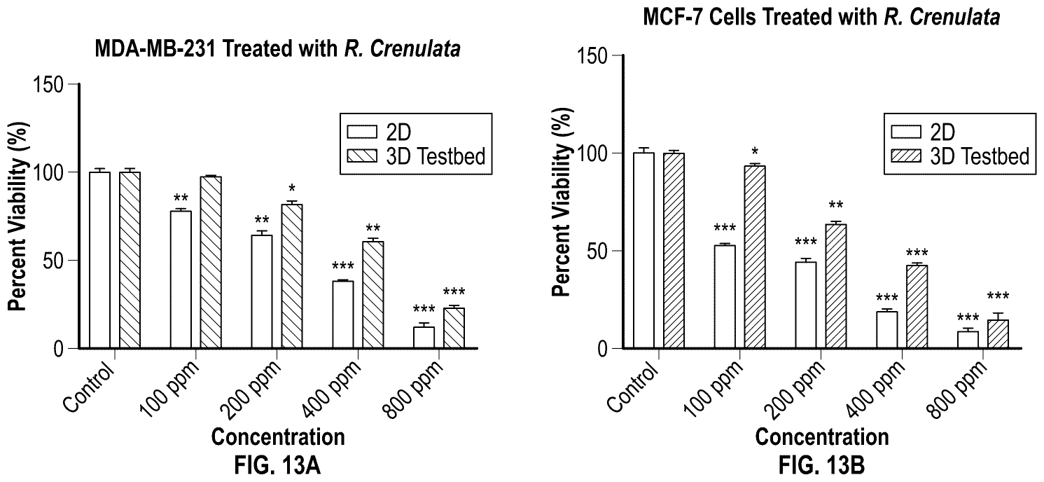

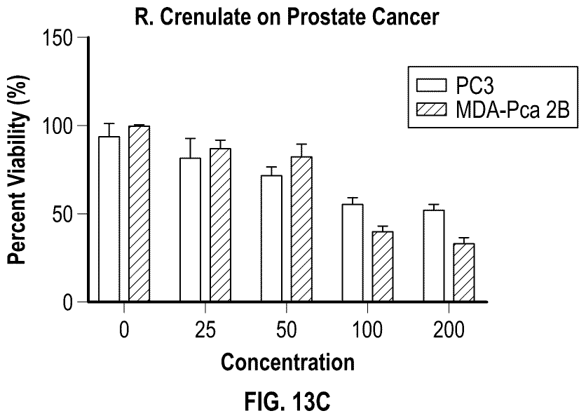

As shown in FIGS. 13A and 13B, the results indicated that R. crenulata reduced the proliferation of 3D MSCs+MCF-7 and 3D MSCs+MM 231 SC, with IC50 drug concentrations of 316.2 ppm and 524.7 ppm, respectively. In contrast, the proliferation of MCF-7 and MM-231 cancer cells in 2D cell cultures were reduced by treating cells with IC50 drug concentration of 100.2 ppm and 291.4 ppm, respectively. Overall, the data indicated that 3D cultured breast cancer cells required higher concentrations of R. crenulata than 2D cultured breast cancer cells to inhibit the growth of 50% cell population. Furthermore, MM-231 BM required higher dosages compared to other sample types.

Similar to the results of R. crenulata that was observed for the MCF-7 and MM-231 breast cancer cells, the cytotoxic effects of R. crenulata was observed for PC3 and MDA-PCa 2B prostate cancer cells. As shown in FIG. 13C, PC3 cells require higher dosages to R. crenulata compared to PCa 2B.

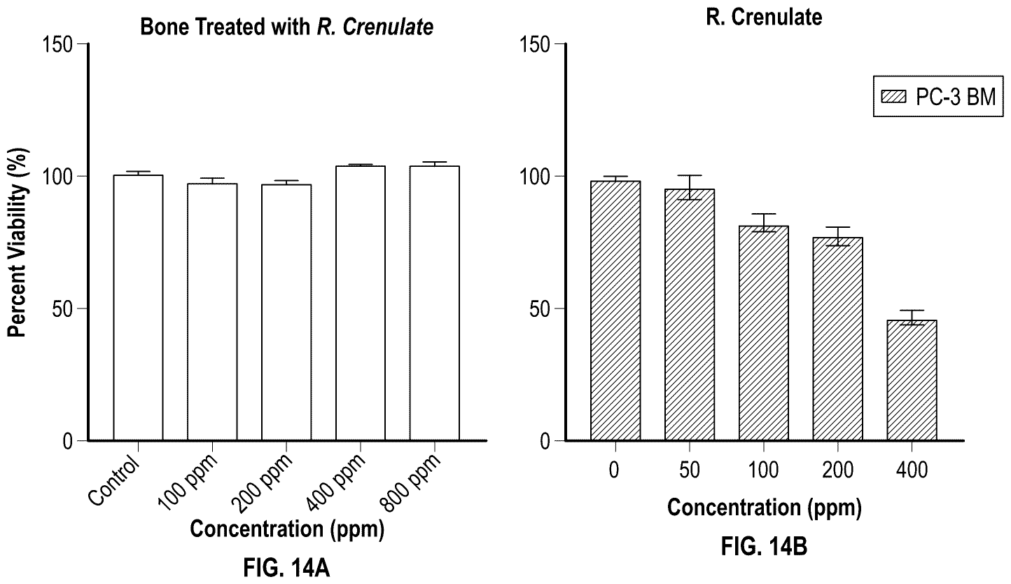

The cytotoxic response of R. crenulata was further evaluated on hMSCs grown on a 3D testbed for 33 days. Bone cells were treated for 24 hours with the same concentrations (100, 200, 400, and 800 ppm) used on the MM 231 and MCF-7 cell cultures. As shown in FIG. 14A, there was no reduction in cell viability found in all concentrations. Furthermore, the cell viability increased significantly when treated with 100 ppm, 200 ppm, 400 ppm and 800 ppm concentrations. Additionally, live/dead staining confirmed that bone cells were unaffected by the treatment of 800 ppm R. crenulata. Further, in FIG. 14B, it is demonstrated that R. crenulata, when treated with 50 ppm, 100 ppm, 200 ppm, and 400 ppm concentrations, can also reduce cell proliferation in Bone-Met (BM) PC3. This demonstrates that higher concentrations of R. crenulata can promote cell proliferation in bone cells at higher concentrations and that bone cells experience no cytotoxic effects with treatment of R. crenulata.

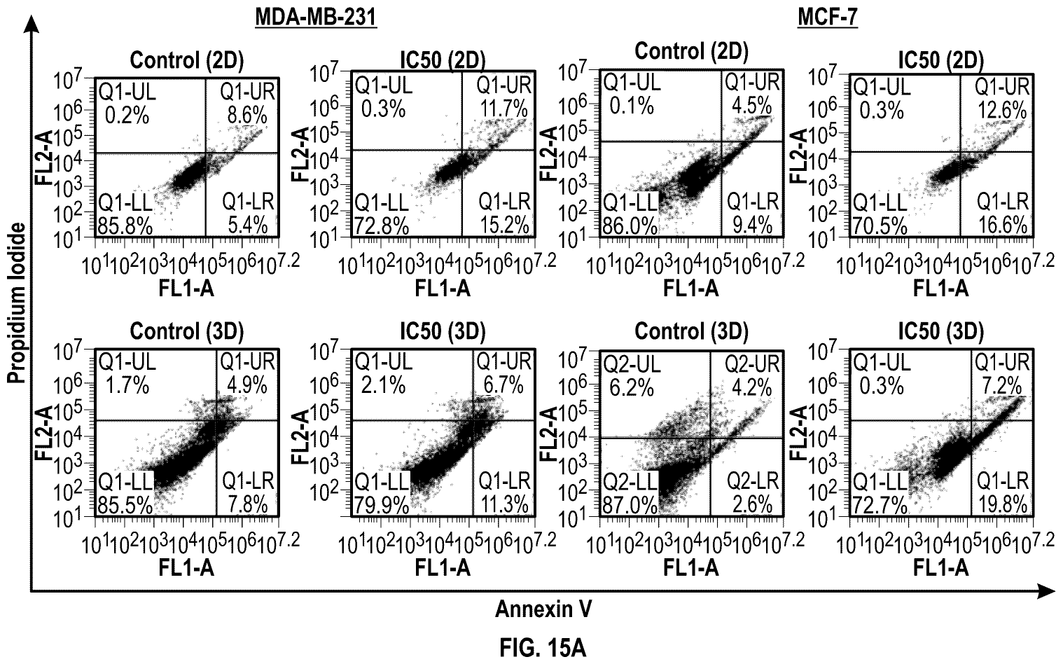

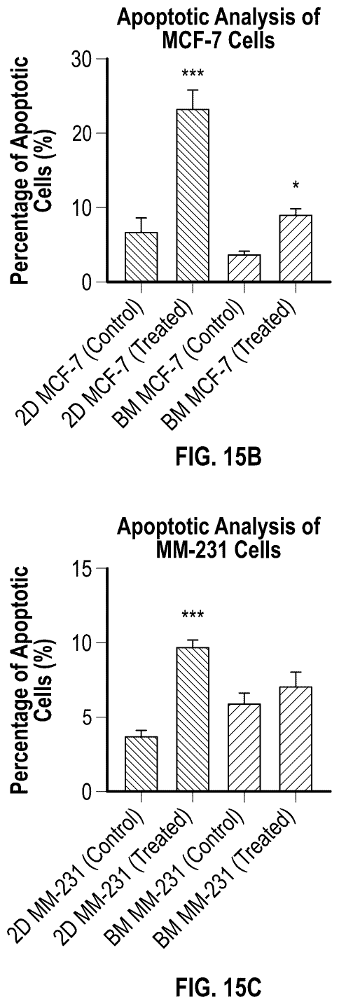

The cytotoxic effects of R. crenulata was observed in 2D cultures of breast cancer cells (MCF-7 and MM231) and 3D sequential culture of breast cancer cells (MCF-7 and MM231) by flow cytometric analysis. Cells were subjected to their respective IC50 concentrations for 12-hours, followed by flow cytometric analysis using Annexin V-FITC apoptosis assay. As shown in FIG. 15A, it was found that treated samples had a high rate of apoptosis compared to non-treated samples, in 2D and 3D cultures. It is shown that there is a slight increase in resistance to apoptosis in 3D sequential cultures of MSCs with breast cancer cells, compared to 2D cultures. The apoptosis assay showed a substantial difference between control and treated samples, for both 2D and 3D cultures.

Further, FIGS. 15B and 15C are bar plots representing the percentages of apoptotic cells for 2D and 3D cultures of breast cancer cells (MM 231 and MC-7). The dollar symbol ($) indicates significant difference in apoptotic percentage between drug treated sample and non-treated samples, in 2D and 3D culture. $ p<0.05 $$ p<0.01 $$$ p<0.001. As shown in FIGS. 15B and 15C, the percentage cell population of early-stage apoptosis induced by R. crenulata was about 8.93% and 5.95% in 2D MM231 and 3D BM MM 231, respectively. Moreover, the early-stage percentage of apoptosis was about 15.9% and 7.7% in 2D MCF-7 and 3D BM MCF-7, respectively, indicating that there was significant resistance to apoptosis in response to R. crenulata in 3D sequential cultures versus 2D cultures.

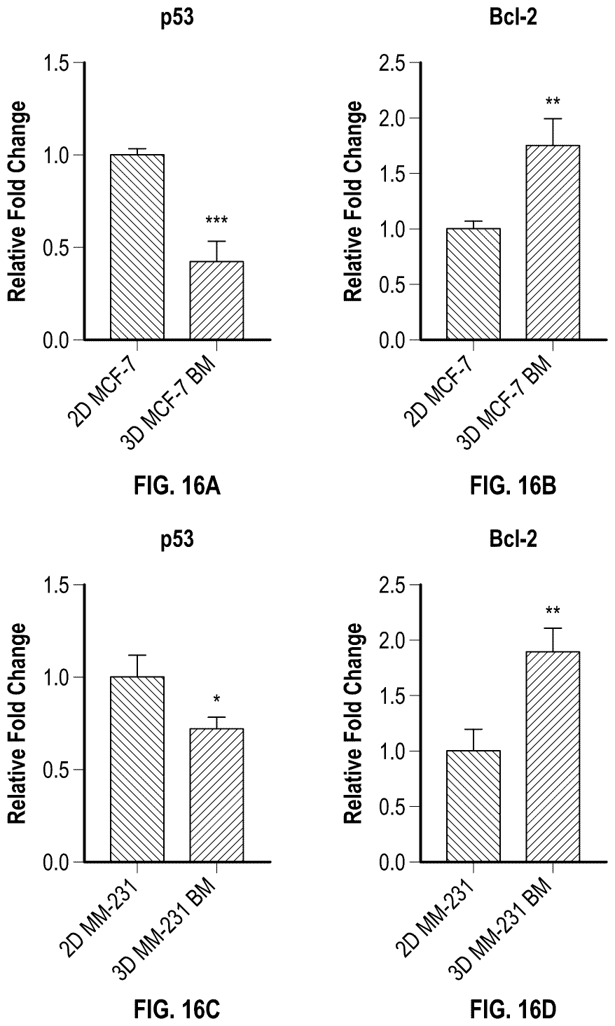

In order to confirm the apoptotic activation by R. crenulata, the expression of anti-apoptotic Bcl-2 and tumor suppressor p53 biomarkers in treated cultures were analyzed. We specifically compared the change in mRNA expression levels between 2D cultures and 3D B.M. cultures after 24 hours of R. crenulata treatment. In MCF-7 breast cancer cells, we observed that MCF-7 BM cultures experienced a ˜2.29-fold decrease in p53 expression compared to 2D MCF-7 cultures (FIG. 16A). Concomitantly, MCF-7 BM cultures experienced ˜1.73-fold increase in Bcl-2 expression compared to 2D MCF-7 cultures (FIG. 16B). A similar trend was observed with MM-231 BM and 2D MM-231 cultures. MM-231 BM cultures experienced ˜1.36-fold downregulation of p53 expression compared to 2D MM-231 (FIG. 16C). In addition, MM-231 BM cultures had a ˜1.91-fold increase in bcl-2 expression compared to 2D MM-231 cultures (FIG. 16D). Overall, breast cancer cells in bone metastatic conditions experience increased resistance to apoptosis after treatment of R. crenulata.

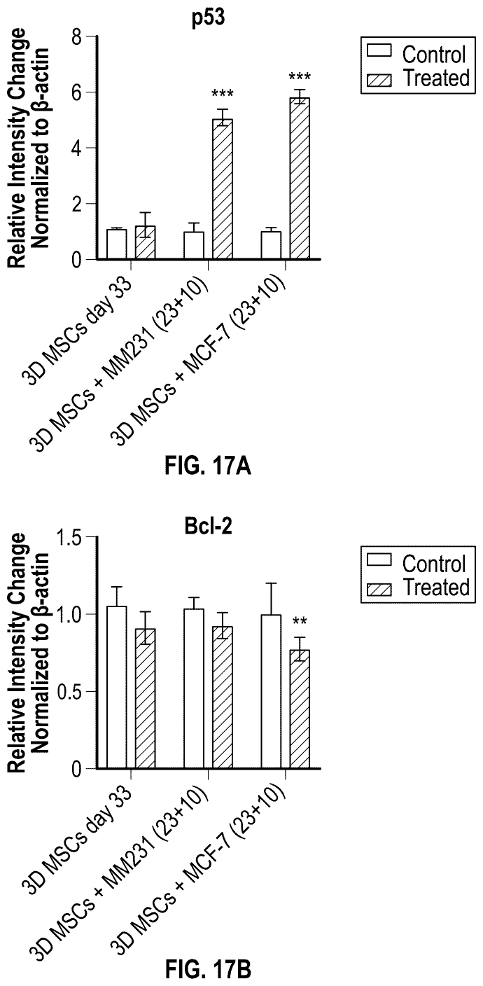

The apoptotic response of bone cells when treated with R. crenulata for 24 hours was additionally evaluated. The relative fold change of treated and non-treated samples for bone and 3D culture of breast cancer cells (MM 231 and MCF-7) was compared. Apoptotic biomarkers experienced no significant changes when healthy bone was treated with R. crenulata. As shown in FIG. 17A, it is shown that there is significant upregulation in treated samples on 3D sequential culture of breast cancer cells in the p53 expression. Again, the asterisk (*) indicates significance of relative expression between treated and non-treated cultures. *p<0.05, **p<0.01 ***p<0.001. However, there was no significant change in treated versus non-treated bone cells. Next, as shown FIG. 17B, the bcl-2 expression was analyzed between 33-day hMSCs (bone) and 3D culture of breast cancer cells (MM 231 and MCF-7). Overall, it was found that there was no significant change in pro-apoptotic and anti-apoptotic markers in treated versus non-treated samples in healthy bone.

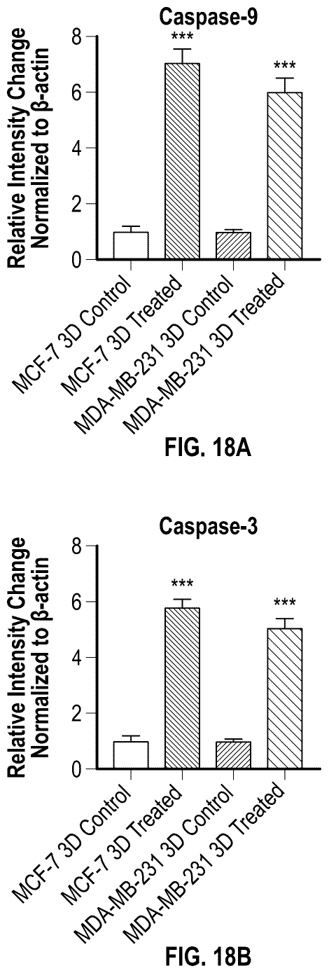

Next, the initiation of apoptosis was confirmed by evaluating the expressions of caspase-9 and caspase-3. R. crenulata activates caspase-9 and caspase-3 in bone metastatic breast cancer (MM 231 and MCF-7). FIG. 18A is a graph illustrating the caspase-9 expression that was measured after 24 hours treatment. The asterisks indicate *p<0.05, **p<0.01 ***p<0.001, or the significant difference in expression of caspase-9 between non-treated cultures and treated culture of breast cancer cells (MM 231 & MCF-7). As shown in FIG. 18A, the results demonstrate that when 3D cultures treated with R. crenulata, caspase-9 expression levels were upregulated significantly, compared to non-treated samples. Furthermore, MCF-7 cells experienced higher fold expression compared to MDA-MB-231 cells. A similar trend related to the expression level of caspase-3 was observed, as shown in FIG. 18B. Both MDA-MB-231 and MCF-7 cells experienced upregulation in caspase-3, with MCF-7 cells experiencing a higher fold expression. Specifically, MCF-7 BM cells experienced nearly ˜6 to 7 times-fold upregulation in caspase-3 and caspase-9 levels, respectively. The MM-231 BM cells experienced nearly a 5-6 times-fold activation in caspase-3 and caspase-9 levels.

In addition to observing the changes in caspase levels, live cell ROS and MtMP imaging of bone metastatic cultures were performed. Samples were treated with IC50 dosages for 12 hours. A decrease in mitochondrial membrane potential intensity after treatment of R. crenulata was observed for both MCF-7 BM and MM231 BM. Concurrently, ROS levels decreased in both bone metastatic cultures. However, treated MCF-7 BM expressed more ROS levels compared to treated MM231 BM.

The aim of this study was to investigate the chemoresistance of bone metastasized breast cancer, when treated with phenolic phytochemically rich extracts of R. crenulata. Breast cancer cells experience altered growth and resistance, when colonizing within the bone marrow. Bone-like ECM formation is observed from osteogenically differentiated MSCs on nano clay scaffolds with enhanced migration of cancer cells in the presence of bone microenvironment. The advantage of using nano-clays in scaffolds is the bone mimetic environments induced, which in addition to the high porosity with interconnected pores, contributes to the high surface area-to-volume ratio in the scaffolds. Additionally, they provide matrix stiffness leading to improved cell-matrix interaction. It has been shown that breast cancer cells grown on bone interface, experience significant resistance to paclitaxel treatment. In previous studies, the effect of R. crenulata has been studied on human breast cancer cells and mouse-derived breast cancer cells. In the present study, the effect of R. crenulata on bone metastasized breast cancer and bone cells. To gauge the viability of R. crenulata as a potential therapy for advanced-stage breast cancer was studied. The IC50 values of breast cancer cells (MDA-MB-231 and MCF-7) grown on 2D TCPS and 3D bone metastatic testbed after 24-hours of R. crenulata treatment were determined. Previously, it was reported that proliferation and invasion was inhibited in MDA-MB-231 and MCF-7 breast cancer cells, in 2D culture and tumorsphere culture, respectively. Here, it was observed that there is an overall increase in cell viability in cancer cells grown on 3D bone metastatic testbed, compared to 2D cultures. Additionally, it was found that MDA-MB-231 breast cancer cells were more resistant to R. crenulata, than MCF-7 breast cancer cells. The calculated IC50 values were higher with MDA-MB-231 cells, compared to MCF-7 cells.

Alternatively, bone cells were treated with R. crenulata for 24-hours. Currently, no studies have been reported on the cytotoxicity of R. crenulata on bone cells. A slight increase in cell viability in higher concentrations was observed, indicating the non-toxic characteristic of R. crenulata. To further investigate the non-toxicity of the treatment, the gene expression levels of pro-apoptotic and anti-apoptotic, p53 and bcl-2, were observed. It was found that there is no significant change in treated and non-treated bone cells. This is further indication that R. crenulata does not affect healthy bone cells.

There is a scarcity of non-toxic therapies available and currently there is no cure for bon metastasis of breast or prostate cancer. It is understood that there are more available therapies for hormonal-positive breast cancer compared to triple-negative breast cancer. The IC50 results presented here suggest that R. crenulata is more suitable for targeting hormonal-positive breast cancer. Next, the ability of R. crenulata to induce apoptosis in 2D and 3D cultures was explored. It was found that treated samples had a higher rate of apoptosis compared to non-treated samples. Furthermore, MCF-7 cells had a higher percentage of cells in the early apoptotic stage compared MDA-MB-231 cells, in both 2D and 3D cultures.

Based on these results, gene expression experiments were conducted to evaluate pro-and anti-apoptotic markers. The tumor suppressor protein, p53, is a protein mainly involved in cell cycle regulation and DNA repair. In response to DNA damage, apoptosis can be triggered by activation of p53. Bcl-2 is part of the anti-apoptotic family of proteins, which are known to be upregulated in chemo-resistant cancer cells. Activation of Bcl-2 proteins leads to inhibition or prevention of cellular apoptosis. In a previous study, it was reported that MCF-7 cells experienced a decrease in ER transcriptional activity, 0-catenin levels, and tumorsphere formation. However, no studies to date have explored R. crenulata apoptotic pathway in bone metastatic breast cancer. The present inventors observed activation of p53 in all treated samples. Furthermore, 2D cultures experienced higher fold expression compared to 3D cultures. The dysregulation of p53 can be linked to chemoresistance in cancer. Additionally, it was found that breast cancer cells grown in 3D bone metastatic testbed had higher fold expression of Bcl-2 compared to 2D cultures. Overall, sequential culture of MSCs with breast cancer cells increased resistance to apoptosis.

Further, the initiation of apoptosis was confirmed by evaluating the expressions of caspases-3 and-9. When apoptosis occurs, cells undergo mitochondrial remodeling and increased ROS production. The stimulation of caspase-9 and effector caspase signifies intrinsic apoptosis. They directly affect the mitochondria, thus regulating/initiating ROS production. Specifically, caspase-9 prevents cytochrome C from accessing complex III in the mitochondria, which allows ROS to be over-produced. Activation of caspase-9 directly activates caspase-3. The activation of caspase-3 allows for cell death to be more efficient. The present results demonstrate that when 3D cultures were treated with R. crenulata, the caspase-3 and caspase-9 mRNA expression levels were upregulated. Concurrently, after treatment, the loss of mitochondrial membrane potential and ROS staining were observed. The activation of caspase-3 inhibits production of ROS and is considered the effector caspase. At bone site, cell death occurs and breast cancer cells experience loss of mitochondrial health, leading to diminished ROS levels. Overall, these results suggest that phytochemically enriched R. crenulata extract can initiate cell death in both primary-site and secondary-site bone metastatic breast cancer.

These testbed studies indicated that the cell viability of breast cancer cells grown both as 2D and 3D cultures, was significantly reduced by Rhodiola crenulata. We also observed that Rhodiola crenulata induced apoptosis in breast cancer cells grown on 3D in vitro testbed, by upregulating pro-apoptotic proteins, p53, caspase-3, and caspase-9. In addition, we observed that breast cancer cells grown at bone metastatic sites on testbeds experienced increased drug resistance, due to upregulation of Bcl-2 expression. Collectively, the data indicates that Rhodiola crenulata induces apoptosis in breast cancer cells at the bone metastatic site and can be pursued further for new therapies to treat bone metastasis of breast cancer.

### Example 3

The potency of different phytochemicals as anticancer agents can be determined by investigating their binding mechanism to protein targets utilizing molecular docking. Molecular docking is an in-silico-chemico biological approach that optimizes the protein-ligand (drug candidate) conformation to compute their minimized binding free energy. Molecular docking is primarily employed to predict the binding orientation of drug candidates against protein targets to predict the drug candidates' affinity and activity. It investigates the protein-ligand interactions along with their strength. Molecular docking has been extensively utilized for anti-breast cancer drug discovery. Phytochemicals have also been investigated for their potential anti-breast cancer activity using docking.

A molecular docking approach was used to evaluate the binding affinities of 123 phytochemicals belonging to Rhodiola (28 compounds), Oregano (22 compounds), and Cranberry (73 compounds) extracts against a significant list of 33 breast cancer protein. These breast cancer proteins have various roles in different stages and mechanisms of breast cancer, including hormone attachment, tumor progression, tumor recurrence, apoptosis, autophagy, metastasis, sternness, etc. However, some proteins can perform dual or multi-directional roles in breast cancer progression.

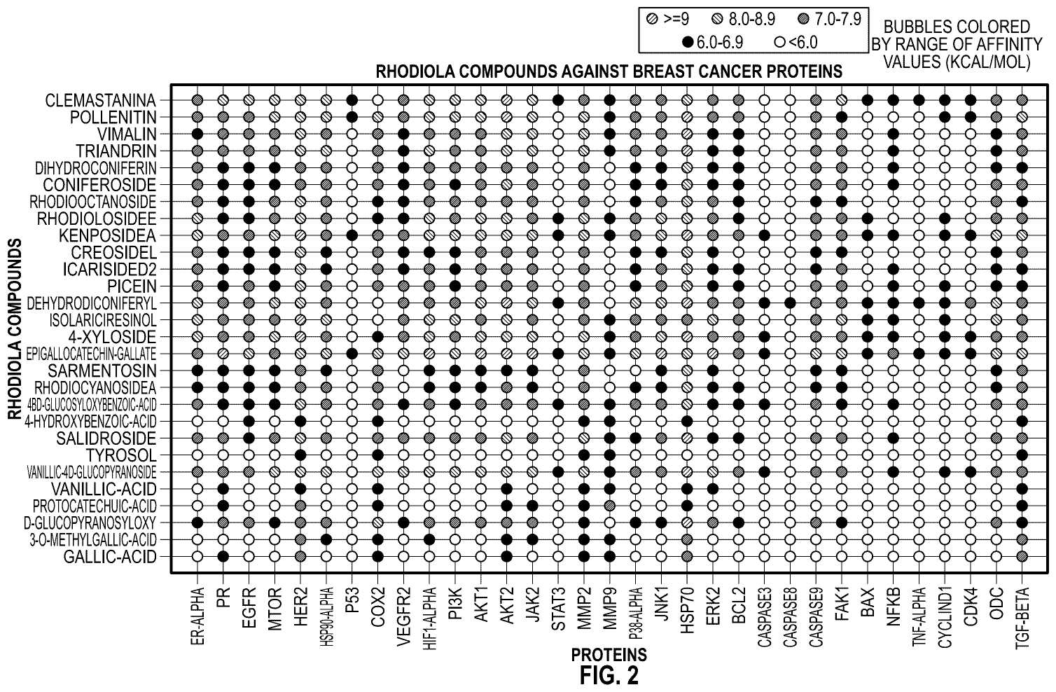

The selected ligands/compounds for molecular docking are phytochemicals derived from Rhodiola, Oregano, and Cranberry extracts. The Rhodiola extract refers to the root extract of Rhodiola crenulata; the Oregano extract refers to the aerial part extract (leaf and flower) of Oreganum vulgare, and the Cranberry extract is from the pomace of the fruit of Vaccinium macrocarpon. These phytochemicals belong to different categories in their structure, i.e., polyphenols, flavonoids, flavonols, terpenoids, carotenoids, etc. FIGS. 2, 3, and 4A-C show the Rhodiola, Oregano, and Cranberry compounds that were docked, respectively.

The molecular docking of selected compounds against breast cancer target proteins was performed using AutoDock Vina 1.1.2. The structures of the selected compounds were obtained in .sdf format from PubChem database. The compound sdf files were converted to 3D pdb format using Open Babel. Next, AutoDockTools (ADT) 1.5.6 was used to prepare the compound pdbqt files from pdb files by merging the non-polar hydrogen, adding the Gasteiger charges and defining the rotatable bonds. These prepared compound pdbqt files were used as ligand input for AutoDock Vina. Additionally, the 3D protein structures were collected from RCSB Protein Data Bank. The protein structures were cleaned by removing co-crystallized ligands, water molecules, ions, etc., to keep a single protein chain using Discovery Studio 2020.

The AutoDockTools were used to prepare the cleaned single-chain protein structure pdbqt file by employing the Gasteiger charges, adding polar hydrogens, and merging non-polar hydrogens. The protein pdbqt files prepared by AutoDockTools were used as protein inputs for AutoDock Vina. The possible active site/ligand binding site on the protein was defined based on the location of the co-crystallized ligand available in literature or by using the UniProtKB database. The search grid was specified for each protein with a spacing of 1 Å. In order to have more consistent docking results, the exhaustiveness parameter was set to 24. Finally, molecular docking was performed utilizing AutoDock Vina 1.1.2 in the Linux platform of the Center for Computationally Assisted Science and Technology (CCAST), a parallel computing facility at North Dakota State University. The proteins were treated as rigid targets during the docking, while the ligands (phytochemicals) were treated as semi-flexible based on their rotatable bonds.

To check the accuracy of docking performed by AutoDock Vina, the undocking and redocking of co-crystallized ligands in several protein structures was performed. These experimentally (X-ray crystallography, cryo-electron microscopy) derived protein-ligand complexes were deposited in the RCSB protein data bank. The selected protein-ligand models were ER-4 Hydroxytamoxifen (PDB ID: 3ERT), PR-Ulipristal acetate (PDB ID: 4OAR), and mTOR-X6K (PDB ID: 4JT6). These co-crystallized ligands were undocked and then redocked again by using AutoDock Vina. If the conformation of redocked ligand was within 2 Å RMSD (root-mean-square deviation) of the experimentally docked ligand, then the docking was considered accurate. The RMSD values were calculated using the PyMOL software. The RMSD values of redocked 4 Hydroxytamoxifen, Ulipristal acetate, and X6K within the E.R., PR, and mTOR cavities were computed to be 1.67 Å, 0.20 Å, and 1.04 Å, respectively. Thus, as all the RMSD values fall under 2 Å, the docking performed by AutoDock Vina can be considered accurate.

The results of molecular docking include protein-ligand binding poses along with their binding affinity in kcal/mol. The higher binding affinity of the protein-ligand complex refers to the higher potency of the ligand as a drug compound. It is essential to consider a threshold value of binding affinity to identify the potent ligand molecules inside different protein cavities. To establish a threshold binding affinity value, the molecular docking of five commercially available breast cancer drug compounds (Alpelisib, Lapatinib, Everolimus, Raloxifene, and Tamoxifen) against their specific protein targets (PI3K, HER2, mTOR, and ER proteins) was performed utilizing AutoDock Vina. The binding affinity of these protein-ligand complexes was utilized to establish the threshold binding affinity. If a ligand binds to the protein with the same binding affinity values as the threshold value or higher, then the ligand was considered a potent compound. These binding affinities are provided in Table 1. The negative value of binding affinity refers to attractive interactions.

TABLE 1
Binding affinities of commercial breast cancer drug
compounds (ligands) against protein targets.

| Protein-Commercial | Binding affinity |
|---|---|
| Drug | (kcal/mol) |
| PI3K- Alpelisib | −8.3 |
| HER2- Lapatinib | −10.7 |
| mTOR- Everolimus | −8.0 |
| ER- Raloxifene | −10.3 |
| ER- Tamoxifen | −9.7 |
| Average | −9.4 |

The average binding affinity of commercial drug compounds was computed as −9.4 kcal/mol. However, to reduce the chance of ignoring a potential phytochemical for breast cancer treatment, all of the 123 Rhodiola, Oregano, and Cranberry compounds with a binding affinity of −9 kcal/mol or higher were evaluated.

The binding affinity values of all Rhodiola, Oregano, and Cranberry compounds against 33 breast cancer proteins are depicted through bubble charts of FIGS. 2-4C. The diameter of the bubble is proportional to the magnitude of the binding affinity value in kcal/mol. For convenience, the bubbles are patterned by the range of affinity values.

FIG. 2 shows that certain compounds of Rhodiola show higher binding affinity toward the breast cancer proteins. Among them, epigallocatechin gallate (EGCG) has been found to be the most potent compound as it shows a higher binding affinity with a range of proteins. Other Rhodiola compounds like vanillic-acid-4D-glucopyranoside, 4-xyloside, and KenposideA have also been observed to bind to different proteins with moderate to high affinity. Almost all the Rhodiola compounds bind to HSP70 with high affinity. Some Rhodiola compounds bind to Akt (Akt1/Akt2) proteins with varying affinity. Statistical analysis was performed by taking the mean of the binding affinity values of a single compound against all 33 breast cancer proteins. This analysis was done for all 28 Rhodiola compounds. Epigallocatechin gallate and Kenposide A were calculated to have highest average values, 8.0 kcal/mol and 7.6 kcal/mol, respectively. Whereas Tyrosol and 4-hydroxybenzoic-acid had the lowest calculated averages, 5.2 kcal/mol, and 5.4 kcal/mol, respectively. 4-xyloside and vanillic-acid-4D-glucopyranoside had a high calculated average of 7.4 kcal/mol.

Among the compounds with high binding affinities, it was found that EGCG had high binding affinities with Akt1 and Akt2, −10.0 (kcal/mol) and −10.1 (kcal/mol), respectively. Akt proteins have a significant role in cell survival, promoting resistance to apoptosis. In prior experimental studies, EGCG reduced Akt regulation in breast cancer cells. EGCG has been studied for different cancer types, activating different protein types. Markers such as JNK and ERK are proteins involved in cancer cell growth, specifically breast cancer. The present docking results indicate that the binding affinities of ERK2 and JNK1 with EGCG are also in the high binding affinity category, with −9.1 kcal/mol and −9.0 kcal/mol binding affinities. Experimental studies have shown EGCG targeting these proteins in breast cancer. The present results indicate which compounds have the best interaction with certain proteins, showing the current docking studies to be a useful tool in screening new potential anticancer compounds.

Alternatively, interactions were found that have high binding affinities, which have not been experimentally investigated. For example, vanillic-acid-4-O-Î²-D-glucopyranoside and vimalin were found to have high binding affinities with HSP70, −10.1 kcal/mol, and −9.3 kcal/mol, respectively. No literature was available on these compounds and their effect on the HSP70 protein. Heat-shock protein 70, upon its activation, can inhibit cancer cell apoptosis. The high binding affinities of these two compounds suggest they are promising anticancer drugs to be investigated. Lastly, it was observed that compounds such as salidroside and pollenitin have weaker binding affinities with HSP70 and Akt2, respectively. Salidroside has been studied experimentally for its efficacy against breast and gastric cancer. In gastric cancer, salidroside has downregulated HSP70 expression. Salidroside is a compound that can be used in targeted therapies based on our results and past experimental studies. On the other hand, pollenitin has not been investigated experimentally. The present docking results show that pollenitin has a stronger binding affinity with HSP70, than with Akt2. Pollenitin is a compound that should be considered for further experimental study on breast cancer.

FIG. 3 shows that Luteolin 7-o-glucoside, Rosmarinic acid, Luteolin-7-o-glucuronide, Apigenin-7-o-glucuronide, and Quercetin 3-o-rutinoside of Oregano exhibit higher binding affinity to a range of proteins, including mTOR, HER2, PI3K, Akt (Akt1/Akt2), JAK2, MMP2, and HSP70. Other potent compounds of Oregano are found to be Eriodictyol, Taxifolin, and Dihydrokaempferol, which bind to several proteins. Compounds from oregano extract are very different than those of Rhodiola extract. Especially those with high binding affinities with similar proteins were different compounds overall.

Compounds from oregano extract are very different than those of Rhodiola extract. Especially those with high binding affinities with similar proteins were different compounds overall. It was found that compounds such as Luteolin-7-o-glucuronide, luteolin-7-O-glucoside, Apigenin-7-o-glucuronide, etc., have the strongest binding affinities. Luteolin-7-o-glucuronide and quercetin-3-O-rutinoside are compounds that registered strong binding affinities with Akt2 and Akt1 proteins. Both their binding affinities were −10.7 kcal/mol and −10.1 kcal/mol, respectively. Various experimental studies have been done on luteolin and quercetin targeting breast cancer proteins. The present docking results are consistent with prior experimental studies. However, the efficacy of compounds such as rosmarinic-acid and eriodictoyl against cancer cells has not been thoroughly investigated. This data suggest that rosmarinic-acid and eriodictoyl have strong binding interactions with HSP70. HSP70 is a significant marker in cancer cell growth and survival. Rosmarinic acid and eriodictoyl are two compounds that should be further investigated as potential anticancer therapies.

Rosmarinic acid also has a good binding affinity (−9.1 kcal/mol) with the protein MMP2. Experimentally, rosmarinic acid was found to downregulate MMP2 expression in MDA-MB-231 breast cancer. The present results propose that rosmarinic acid is better suited to target HSP70 (−9.9 kcal/mol) than MMP2. HSP70 is an emerging target for targeted cancer therapy. The expression of HSP70 is related to poor prognosis in different types of cancer. Inhibiting HSP70 expression in cancer will block resistance to apoptosis. In contrast, the interaction between eriodictoyl and JAK2 (−9.0 kcal/mol) is a slightly weaker interaction (although within the high binding affinity range) that has not been investigated experimentally. JAK2 is a protein involved in cell growth and proliferation. Many of the oregano compounds seem to interact strongly with HSP70.

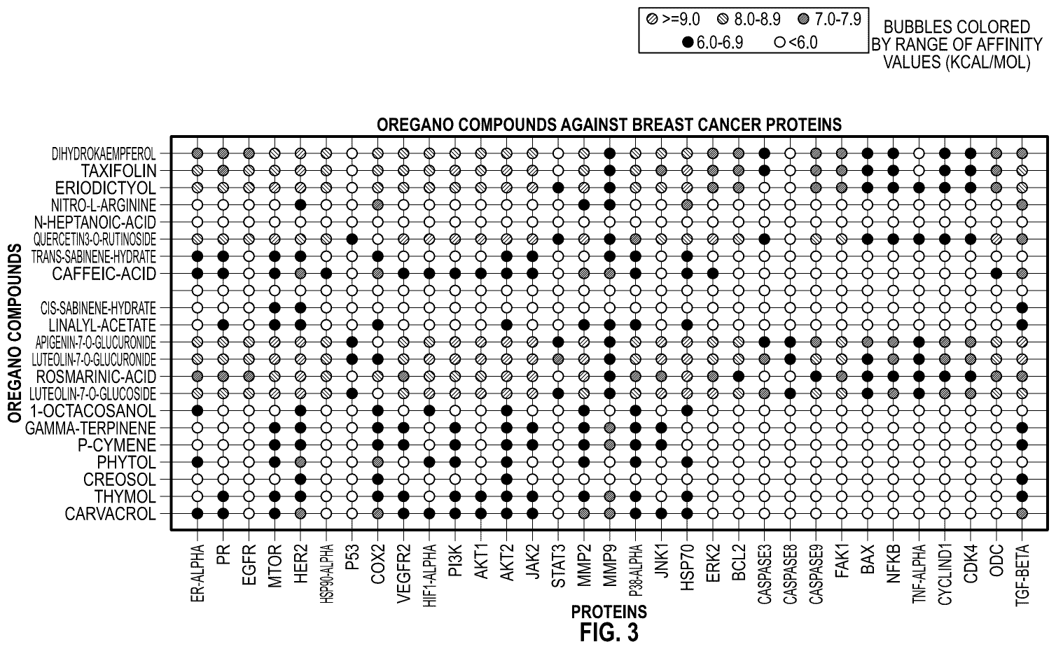

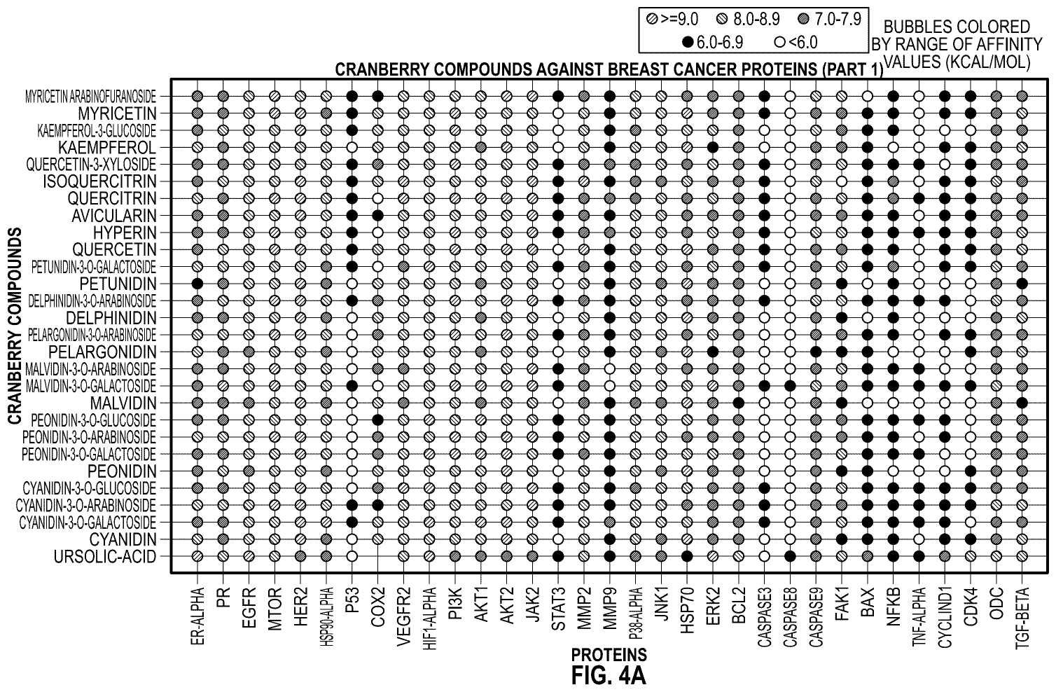

FIGS. 4A-C show the bubble chart of the binding affinity of the protein-ligand complexes for cranberry extract and breast cancer proteins. Phytochemicals from the cranberry extract are slightly different from those of Rhodiola and very different from those of Oregano. Cranberry compounds such as catechin-gallate and gallocatechin-gallate have stronger interactions with some of the proteins. These compounds are derivatives of EGCG and have similar effects. Our results show that catechin-gallate had the strongest interaction with Akt2, −10.8 kcal/mol, and that folic-acid and procyanidinA2 had strong interactions with HSP70 and P.R., (−10.6 kcal/mol and −10.4 kcal/mol), respectively. Statistical analysis was performed to identify the effective compounds.

When the protein-compound interactions at the lower end of the high-affinity range are observed, potential compounds of interest are identified, such as EGCG and Quercitrin. For example, the interaction between EGCG and HIF1-alpha had a binding affinity value of −9.2 kcal/mol. The potential of EGCG to inhibit HIF-1α activation in breast cancer has been studied. In another case, it was found that the interaction between Quercitrin and JAK2 is −9.3 kcal/mol. Quercitrin is a glycosidic derivative from Quercetin. Quercetin is a typical flavonoid-phytochemical found in fruits and vegetables. Quercetin is a phytochemical that has been used against different cancer types. In literature, Quercetin has been shown to downregulate JAK/STAT signaling in cholangiocarcinoma. JAK/STAT signaling is a pathway crucial to cancer progression as an intrinsic tumor driver of cancer growth/metastasis. The present results suggest other phytochemicals are also better suited to target the JAK2 protein, for example, the interaction between Sitogluside and JAK2 to be −9.3 kcal/mol. However, no literature has been reported regarding Sitogluside and its effect on JAK protein or breast cancer. Though, its binding affinity is as strong to JAK2 as Quercitrin.

Overall, this Example demonstrates the viability of these modeling studies to correctly identify phytochemicals shown to suppress cancer or have strong interactions with breast cancer proteins. EGCG exhibits the most potent behavior among the Rhodiola compounds as it binds with several breast cancer proteins. The Oregano compounds showing higher potency with several breast cancer proteins are Luteolin, Apigenin, Quercetin, and Rosmarinic acid derivatives. The cranberry compounds that show broad affinity with breast cancer proteins include Gallate compounds, including EGCG, procyanidine compounds, quercetin, sitogluside, among others. The results show that the molecular docking-based studies conducted have a strong potential to identify phytochemicals from the Rhodiola, Oregano and Cranberry extract likely to suppress breast cancer, leading to new anticancer compounds.

### Example 4

The cytotoxicity of phenolic compounds Origanum vulgare and Vaccinium macrocarpon were further evaluated on bone metastatic and non-metastatic breast cancer.

### Materials and Methods

Human breast cancer cell lines MDA-MB-231 (MM 231) and MCF-7 cell lines, along with Dulbecco's Modified Eagle Medium (DMEM) and Eagle's Minimum Essential Medium (EMEM), Fetal Bovine Serum, and Penicillin/Streptomycin were purchased from ATCC (Manassas, Virginia, USA). Human mesenchymal stem cells (hMSCs) and MSCGM bulletkit medium were purchased from Lonza (Walkersville, MD, USA). Polycaprolactone (PCL) (average Mn 80,000), 5-aminovaleric acid, calcium chloride (CaCl2)), sodium phosphate (Na2HPO4), and 1,4-dioxane were purchased from Sigma Aldrich (St. Louis, MO, USA). Direct-zol RNA MiniPrep kit was purchased from Zymo Research (Irvine, CA, USA). Gibco™ human recombinant insulin, Applied Biosystems™ Fast SYBR Green, and Alamar blue cell viability reagent were purchased from Invitrogen (Waltham, MA, USA). ApoScreen® Annexin V-FITC kits were purchased from Southern Biotech (Birmingham, AL, USA). M-MLV reverse transcriptase kit was purchased from Promega (Madison, WI, USA). Na-MMT clay was obtained from Clay Minerals Repository at the University of Missouri. V.M. extract was purchased from Decas Cranberry products Inc. (Carver, MA). O.V. extract was purchased from Barrington Nutritionals (Harrison, NY).

### Preparation of Phytochemically-Enriched Plant Extract Solutions

Initially, a stock solution (1000 ppm) of Origanum vulgare (O.V.) and Vaccinium macrocarpon (V.M.) was prepared by dissolving 0.01 g each of the herbal extract (dry powder) in 100 mL of 10% ethanol solution. Next, the phytochemically-enriched therapeutic solution was passed through a 0.22 μm filter for sterilization. Further, the stock solutions were further diluted in serial dilutions using serum-free media.

### Preparation of PCL/In Situ HAPclay 3D Scaffolds

Initially, the procedure begins with modifying Na-MMT clay with 5-amino valeric acid to increase the d-spacing of the clay. Next, hydroxyapatite (HAP) was biomineralized within the intercalated nano-clay galleries, creating in situ HAP clay. Further, the PCL was incorporated with 10% in situ HAPclay and dissolved in 1,4-dioxane. The solution was stirred and poured into cylindrical molds. PCL/in situ HAP clay scaffolds were prepared utilizing the freeze-drying method. The dimensions of the scaffolds were cut to 12 mm in diameter and 3 mm in thickness and used for experiments.

### Cell Culture and Cell Seeding

Media for MDA-MB 231 cells was composed of 90% DMEM, 10% FBS, and 1% Penicillin-Streptomycin (P/S). Media for MCF-7 cells was composed of 90% Eagle's Minimum Essential Medium (EMEM), 10% FBS, 0.01 mg/mL human recombinant insulin, and 1% P/S. Human mesenchymal stem cells were cultured using an MSCGM bullet kit medium. All cultures were maintained at 37° C. and 5% CO₂ in a humidified incubator. Breast cancer cell lines were used from passages 4-8, and hMSCs were used from passages 2-5. For 2D cultures, 5×10⁴ breast cancer cells were cultured on tissue culture polystyrene (TCPS). For 3D Bone-metastatic (BM) cultures, scaffolds were first sterilized under UV light for 45 minutes and then immersed in 70% ethanol for 24 hours. Next, they were washed in PBS twice for 12-hour intervals and stored in a CO2 incubator, immersed in culture medium. Then, scaffolds were seeded with 1×10⁵ hMSCs per scaffold and kept for 4 hours before adding culture medium. Cell-seeded scaffolds were cultured for 23 days for bone-like extracellular matrix (ECM) formation. Next, 1×10⁵ breast cancer cells were seeded per scaffold on bone scaffolds and cultured for ten days.

### Cell Viability Assay

Both 2D and 3D bone metastatic cultures of breast cancer cells and bone cultures were serum-starved for 24 hours prior to treatment. Subsequently, all cultures were treated with different concentrations (0, 100, 200, 400, 800 PPM) of O.V. and V.M. solutions for 24 hours. Afterward, the cell viability of treated and untreated (control) samples was measured using Alamar blue cell viability reagent, following the manufacturer's protocol. Half maximal inhibitory concentration (IC₅₀) values for 2D and 3D-BM cultures were calculated using Graph Pad Prism (v7.04) using nonlinear regression analysis.

### Live/Dead Assay

Live/dead assay was performed to evaluate the viability of MM231 and MCF-7 bone metastatic cultures and normal bone cultures. Samples imaged were untreated and treated with IC50 concentrations with O.V. and V.M. Cells seeded on the different scaffolds were stained with a live/Dead™ Cell Imaging Kit (Thermofisher Scientific, Germany) according to the manufacturer's protocol. Live/dead solution was prepared by mixing the Calcein AM solution and BOBO-3 iodide and diluting it to a working concentration with PBS. Next, treated and untreated samples were introduced with both live stain (Calcein AM) and dead stain (BOBO-3 iodide)). Lastly, scaffolds were incubated in the live/dead solution for 15 minutes at room temperature. Samples were imaged using JPK Nanowizard Bio-AFM confocal system using FITC/TRITC filters.

### Flow Cytometric Analysis of Apoptosis

For flow cytometric analysis, 2D cultures and 3D-BM cultures were treated for 12 hours with their respective IC₅₀ concentrations. Then, treated and untreated cells were harvested and washed in cold PBS 3× times. The cells were resuspended in cold annexin binding buffer to a concentration of 1×10⁶ cells/mL, then labeled with Propidium Iodide (PI) and Fluorescein isothiocyanate (FITC)-conjugated Annexin V. Samples were run using BD Accuri C6 Flow cytometer and processed using FlowJo software.

### Reactive Oxygen Species (ROS) Assay

Live bone-metastatic cultures of MM231 and MCF-7 were stained following the manufacturer's protocols. Briefly, samples were treated with O.V. and V.M. for 12 hours, then rinsed with PBS prior to adding DCFDA solution. DCFDA solution was diluted and added to samples in the dark for 45 minutes at 37° C. After, samples were washed with 1× buffer and then imaged. Live cell imaging was performed using JPK nanowizard bio-AFM confocal system with FITC filter.

### Mitochondrial Membrane Potential (MtMP) Assay

Manufacturer's protocols were followed for live cell imaging. Bone metastatic cultures of MM231 and MCF-7 were treated with O.V. and V.M. for 12 hours. After, samples were washed with PBS. Next, 200 nM of TMRE labeling solution was added to all samples. Samples were placed in CO₂ incubator for 20 minutes. Samples were imaged using JPK nanowizard bio-AFM confocal system, using the orange-red filter.

### Gene Expression Studies

Before breast cancer cells were seeded on the bone scaffold, hMSCs were cell cycle-arrested with 10 μg/mL of Mitomycin C. Both 2D, 3D-BM, and bone cultures were treated with IC50 drug concentration. After 24 hours of treatment, total RNA was isolated using Direct-zol RNA miniprep kit. Isolated RNA was reverse transcribed to synthesize cDNA using M-MLV reverse transcriptase (Promega), random primers, and thermal cycler (Applied Biosystems). Finally, Real-Time Polymerase Chain Reaction (RT-PCR) was performed using a thermal profile with a holding stage (2 min at 50° C., 10 min at 95° C.) and a cycling stage (40 cycles of 15 s at 95° C., and 1 min at 60° C.) on 7500 Fast Real-Time System (Applied Biosystems). Differences in mRNA expression of p53, Bcl-2, caspase-3, and caspase-9, between treated and untreated cultures were measured. Gene expressions were normalized to P-actin. Target gene expressions were calculated using the comparative Ct method (2^{-ΔΔCt}).

### Statistical Analysis

The data presented was calculated as mean±standard deviation (n=3). Using one-way ANOVA, the statistical significance/p-values among multiple comparisons were determined, followed by post hoc Tukey test. Statistical differences between treated and untreated groups were determined to be significant utilizing an unpaired Student's t-test, using GraphPad Prism v7.04. and at 95% probability level (p<0.05).

### Results

Cell viability assay was employed to observe the cytotoxic effects of O.V. and V.M. treatment on 2D and 3D BM cultures. Cells were treated with 100, 200, 400, and 800 ppm of both phytochemically-enriched plant extracts for 24 hours. The IC50 concentration was determined from the dose-response study using curve fitting. Treatments of O.V. and V.M. induced various responses in 2D and BM cultures. After treatment of O.V., BM MM231 and BM MCF-7 experienced a reduction in viability with increasing dosages. However, BM cultures had a higher percentage of viable cells compared to 2D cultures. The IC50 drug concentrations of BM MM231 and BM MCF-7 are 767.4 ppm and 590.3 ppm, respectively (FIGS. 5A and 5B).

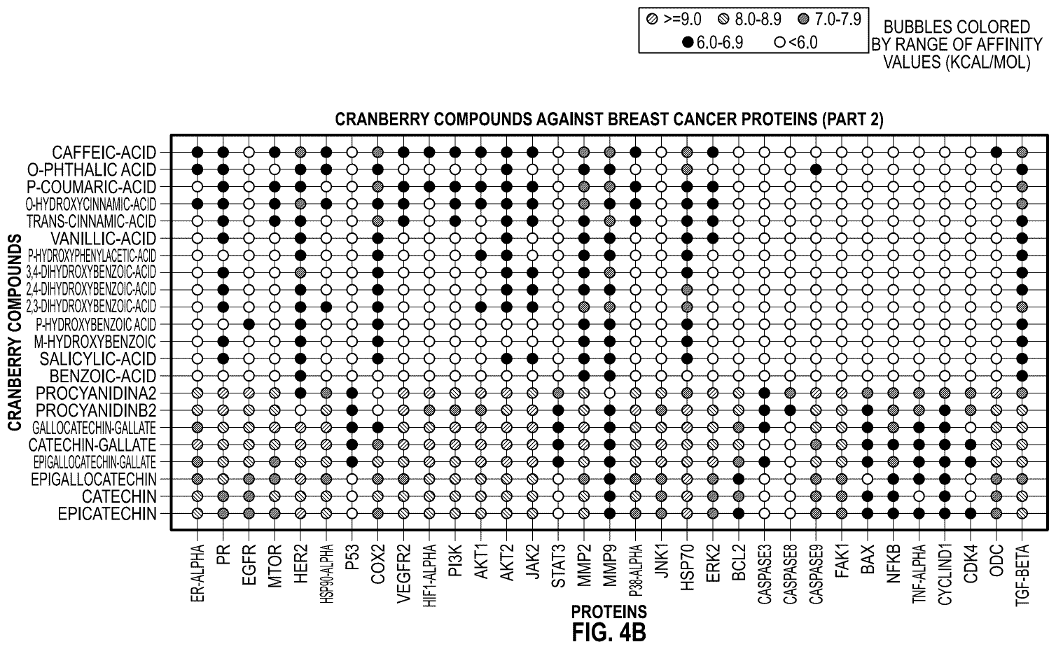

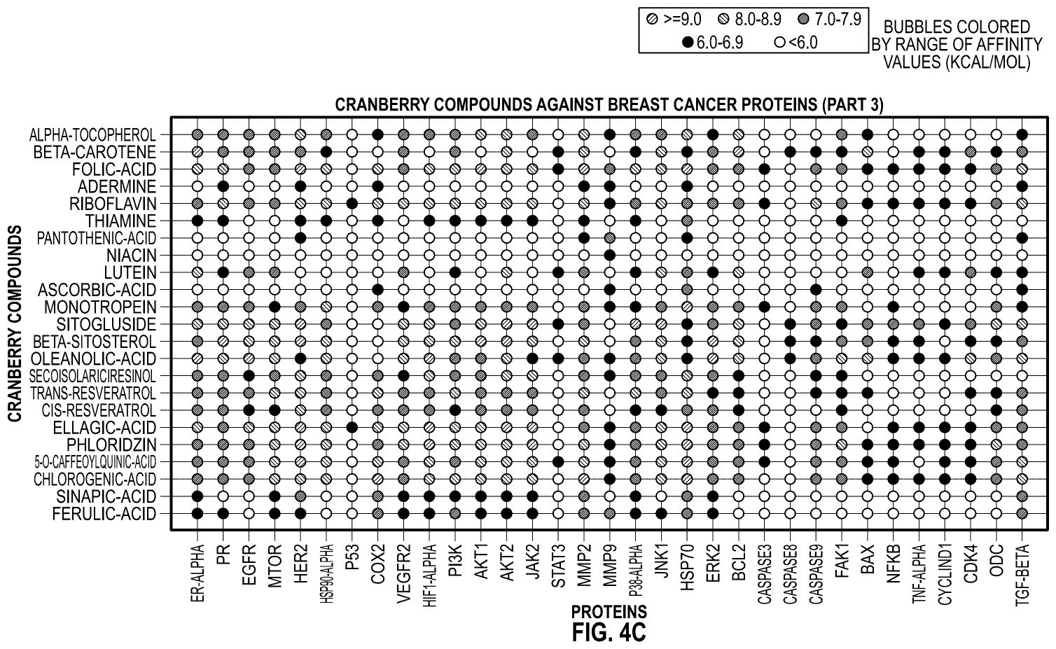

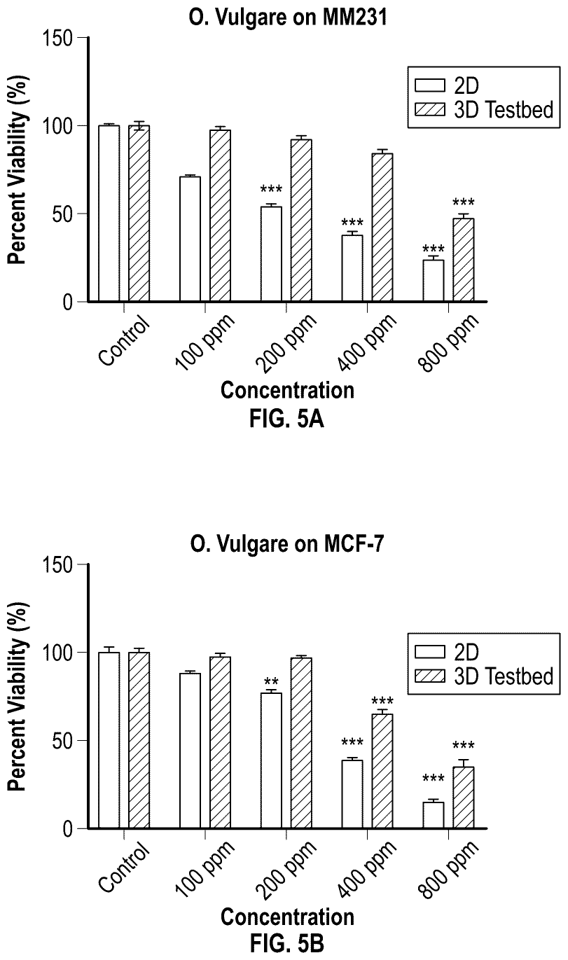

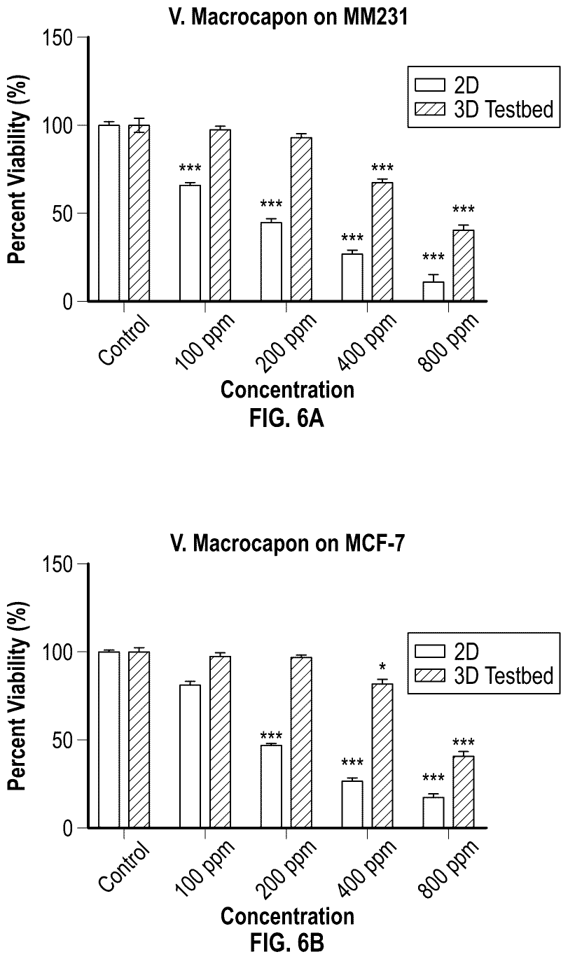

In contrast, the IC50 concentration of MCF-7 and MM231 cancer cells in 2D cell cultures were reduced in comparison, with values of 310.7 ppm and 240.2 ppm, respectively (FIGS. 5A and 5B). Alternatively, post-treatment of V.M., BM MM231 and BM MCF-7 experienced opposite responses, compared to O.V. treatment. BM MCF-7 required a higher IC5 dosage of V.M. compared to BM MM231 even though BM cultures had a higher percentage of viable cells compared to 2D cultures. The IC50 drug concentrations of BM MM231 and BM MCF-7 are 660.8 ppm and 735.7 ppm, respectively (FIGS. 6A and 6B). In contrast, the IC50 concentrations of MCF-7 and MM231 cancer cells in 2D cell cultures were smaller in comparison, with values of 194.2 ppm and 185.5 ppm, respectively (FIGS. 6A and 6B). Overall, these results indicate that 3D BM cultured breast cancer cells required higher concentrations of O.V. and V.M., compared than 2D cultured breast cancer cells. Furthermore, treatment with O.V. was more effective against BM MCF-7, whereas V.M. was more effective against BM MM231.

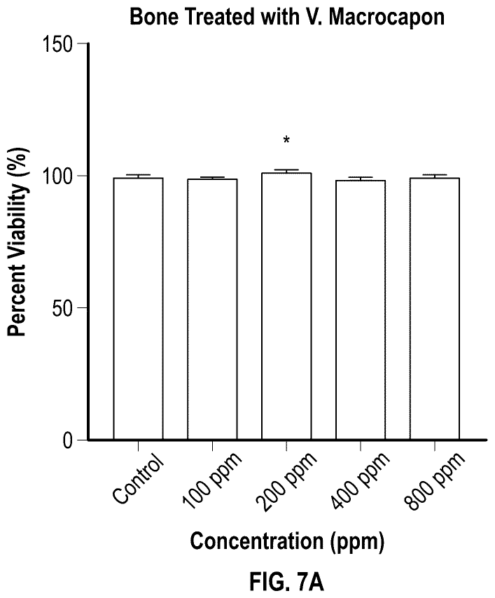

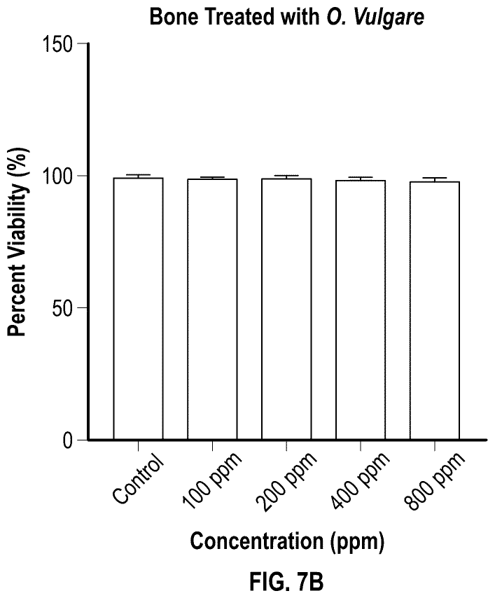

The cytotoxic response of bone (hMSCs grown on a 3D testbed for 33 days) was evaluated. Bone cells were treated for 24 hours with 100 ppm, 200 ppm, 400 ppm, and 800 ppm concentrations of both O.V. and V.M. We observed that there was no reduction in cell viability found in all concentrations for both treatments (FIGS. 7A and 7B). Furthermore, live-dead images confirmed that bone cells were unaffected after 24 hours of treatment of 800 ppm of both extracts. Overall results indicate that both phytochemical-enriched plant extracts are non-lethal toward normal bone cells.

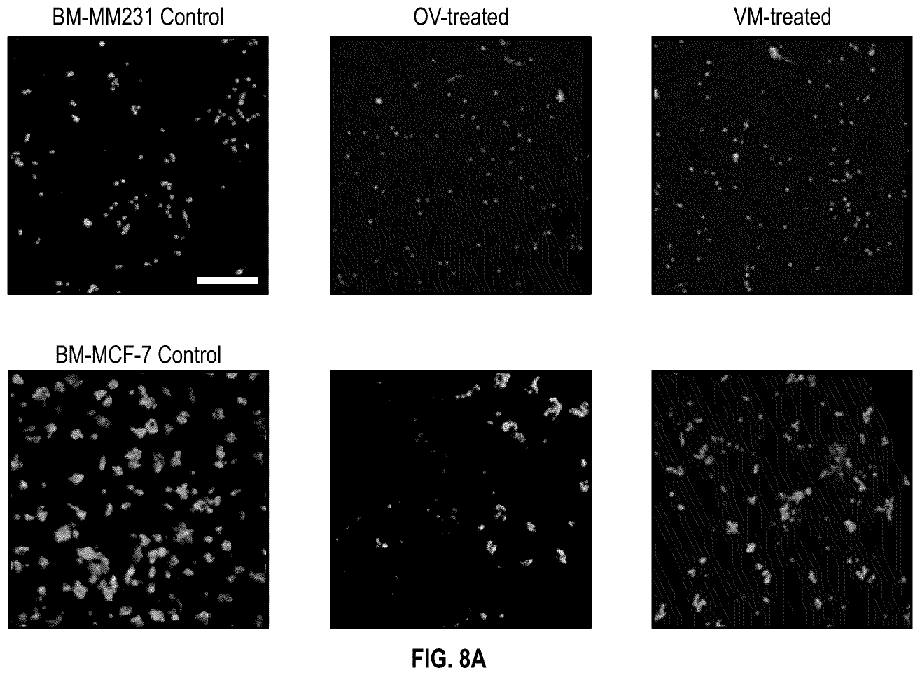

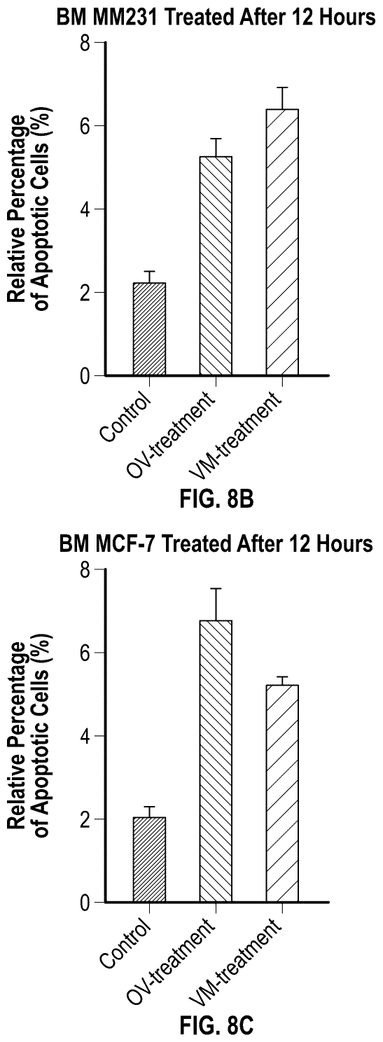

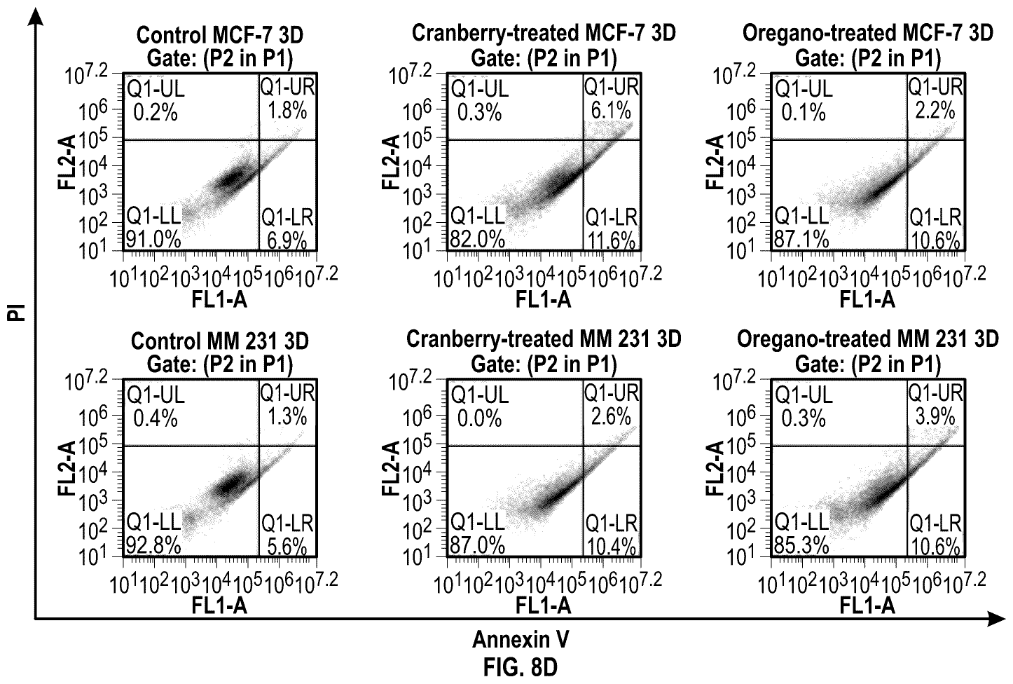

BM breast cancer cells were treated with their respective IC50 dosages of O.V. and V.M. to confirm the resulting cytotoxicity (FIG. 8A). Live dead imaging indicated that post treatment the percentage of live cells decreased, and dead cells percentage increased. Images indicated that BM MM 231 had a similar number of live cells (green stain) after O.V. and V.M. treatment. Meanwhile, BM-MCF-7 had more dead cells after O.V. treatment. Overall, treatment of O.V. and V.M. induced death in BM breast cancer cells.

Additionally, breast cancer cell BM cultures were subjected to their respective IC50 concentrations of both O.V. and V.M. for 12 hours. Flow cytometric analysis using annexin V and propidium stain to evaluate the percentage of cells that become apoptotic. It was found that treated samples had an increased rate of apoptosis compared to non-treated samples in 3D BM cultures (FIGS. 8B and 8C). As indicated, the apoptosis assay showed a significant difference between control and treated samples for 3D BM cultures. The percentage of the cell population that became apoptotic, induced by O.V. and V.M., was ˜5.33% and ˜6.25% in 3D BM MM-231, respectively. The early-stage percentage of apoptosis was ˜6.32% and ˜5.7% in 3D BM MCF-7, respectively, indicating a significant increase in apoptotic cells to both O.V. and V.M. in 3D BM cultures.

Apoptosis in bone-metastatic breast cancer cells is additionally shown in FIG. 8D. The representative fluorescent channel dot plot of FIG. 8D is an analysis of 2D and 3D sequential cultures of breast cancer cells (MM 231 and MCF-7) showing double staining of Annexin V and Propidium Iodide. The cells were treated for 12 hours with their respective IC50 concentrations. The cells were double stained with Annexin V (x-axis) and Propidium Iodide (y-axis).

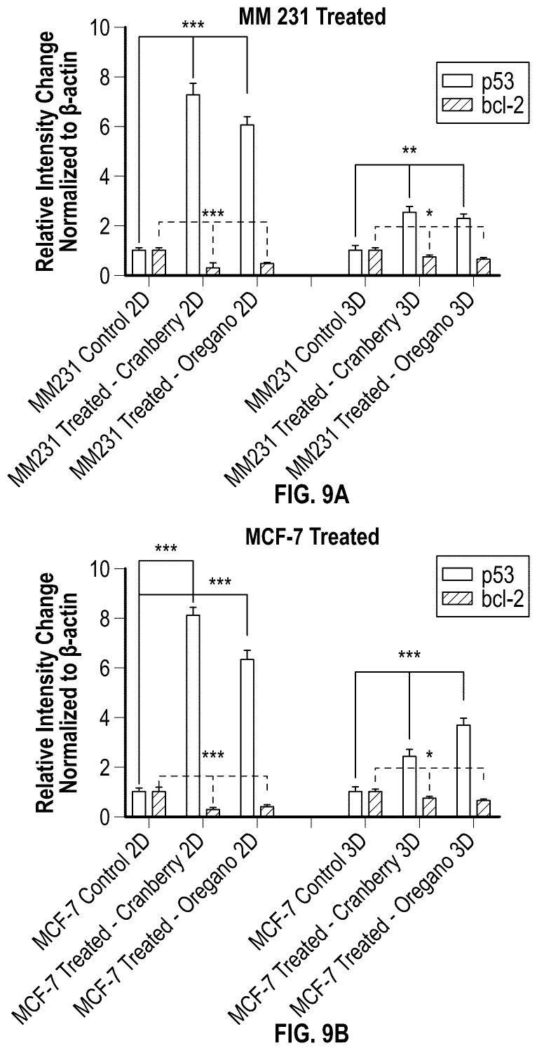

To confirm the cell death initiation, we analyzed the expression of anti-apoptotic Bcl-2 and tumor suppressor p53 biomarkers in treated cultures. We compared the fold-change of mRNA expression levels between treated and untreated after 24 hours of O.V. and V.M. treatment. In MCF-7 breast cancer cells, we observed that 2D cultures experienced a ˜5.99-fold and ˜7.13-fold increase in p53 expression compared to untreated cultures, respectively (FIG. 9A). MCF-7 BM cultures experienced ˜2.15-fold and ˜1.78-fold increase in p53 expression, compared to untreated cultures. Alternatively, Bcl-2 levels were observed in treated 2D MCF-7 cultures and BM MCF-7 cultures. In MCF-7 breast cancer cells, we observed that 2D cultures experienced a ˜3.31-fold and ˜4.07-fold decrease in Bcl-2 expression, compared to untreated cultures, respectively (FIG. 9B). MCF-7 BM cultures experienced ˜1.53-fold and ˜1.75-fold decrease in bcl-2 expression, compared to untreated cultures. A similar trend was observed with MM231 2D and BM MM231 cultures. After 24 hours of O.V. and V.M. treatment, MM231 2D cultures experienced a ˜5.71-fold and ˜6.22-fold increase in p53 expression compared to untreated cultures, respectively (FIG. 9B). While MM231 BM cultures experienced ˜1.84-fold and ˜1.99-fold increase in p53 expression, compared to untreated cultures. Additionally, Bcl-2 levels in treated 2D MM231 cultures and BM MM231 cultures were observed. We observed that 2D cultures experienced a ˜3.125-fold and ˜3.74-fold decrease in Bcl-2 expression, compared to untreated cultures, respectively (FIG. 9B). Whereas MCF-7 BM cultures experienced ˜1.19-fold and ˜1.23-fold decrease in bcl-2 expression, compared to untreated cultures. Overall, breast cancer cells in bone metastatic conditions experience increased resistance to apoptosis after treatment of Origanum vulgare and Vaccinium macrocarpon.

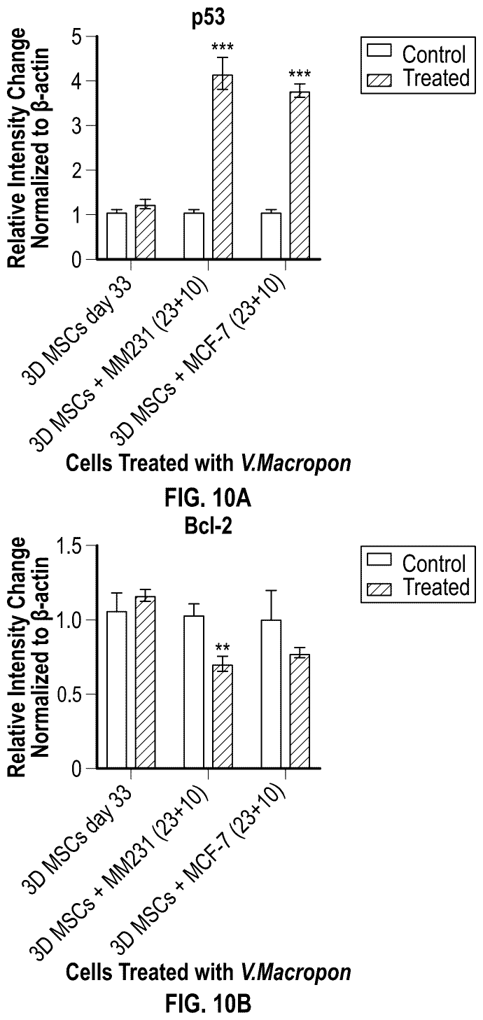

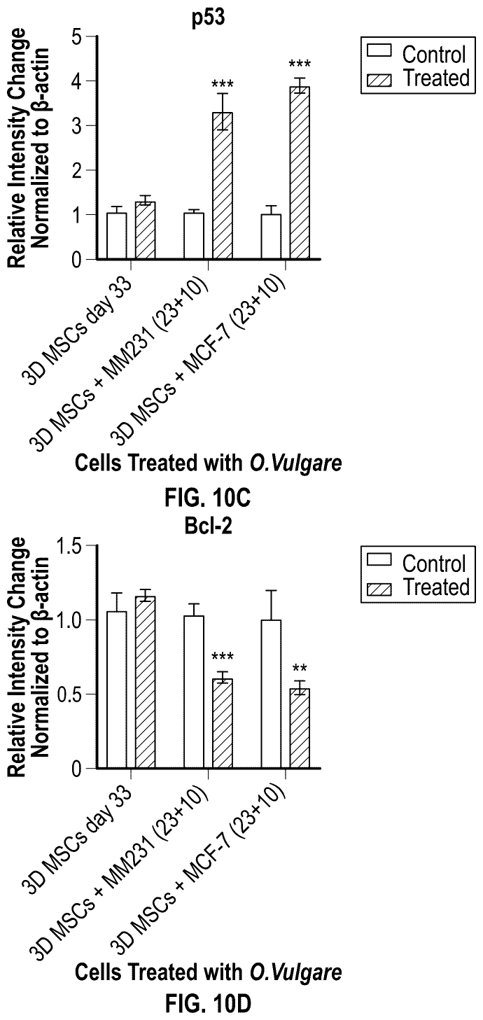

We evaluated the apoptotic response of bone cells when treated for 24 hours and compared the relative fold change of treated and non-treated samples for bone and 3D BM culture of breast cancer cells, MM 231 and MCF-7. Samples were treated with O.V. and V.M. for 24 hours. Firstly, Bcl-2 expression was compared between bone and 3D cultures of breast cancer cells. In healthy bone, we observed no significant change in either treated conditions versus non-treated samples (FIGS. 10A-D). The same trend was observed, where there is a significant upregulation of p53 expression in treated samples on 3D BM culture of breast cancer cells. However, no significant change in treated versus non-treated bone cells was observed (FIGS. 10A-D).

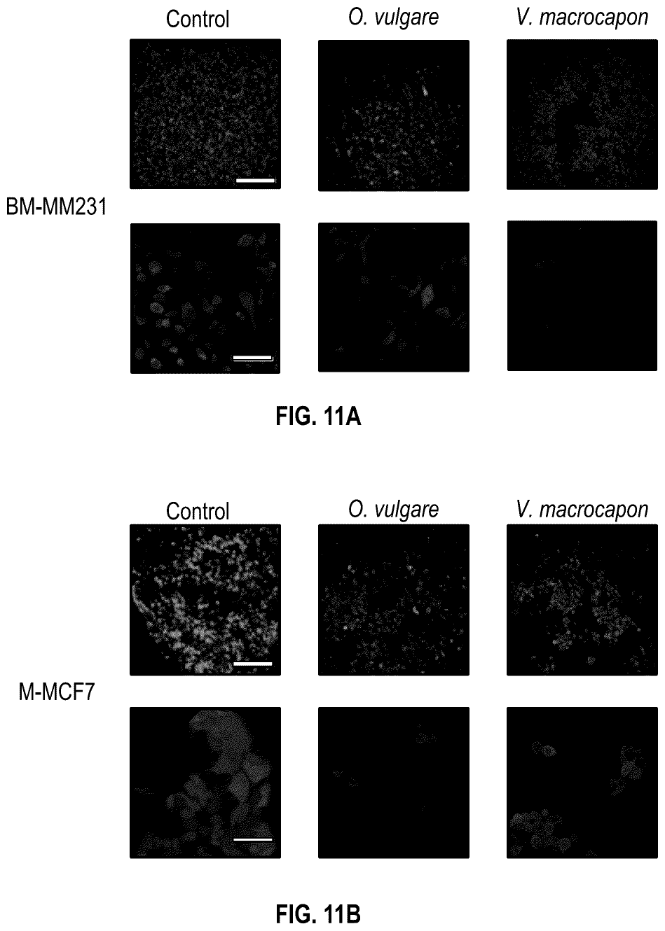

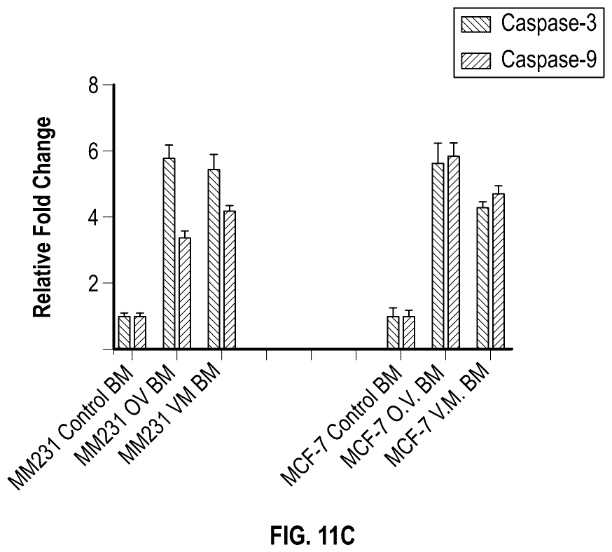

Live cell imaging of bone metastatic cultures was performed to evaluate the health of cells after treatment of phytochemically enriched plant extracts. Both ROS levels and mitochondrial membrane potential of BM cultures were determined after treatment of both plant extracts: O.V. and V.M. We observed the mitochondrial membrane potential staining reduced in BM MM231 cultures after treatment of the three PE-plant extracts (FIG. 11A). Consequently, we observed the ROS levels to be unchanged after the treatment in BM MM231. Alternatively, BM-MCF7 cultures experienced reduced mitochondrial membrane potential (FIG. 11B). Live cell staining also showed reduced ROS levels after treatment. Consecutively, we confirmed the initiation of apoptosis by evaluating the expressions of caspase-9 and caspase-3. Our results show that when 3D BM cultures were treated with O.V. and V.M., caspase-3 expression levels were upregulated significantly in treated samples compared to non-treated samples (FIG. 11C). Furthermore, BM MCF-7 cells experienced higher fold expression compared to BM MM231 cells. We observed a similar trend related to the expression level of caspase-9 (FIG. 11C). However, MM231 expressed more caspase-9 when treated by V.M., while MCF-7 expressed more caspase-9 when treated by O.V. Specifically, MCF-7 BM cells treated with O.V. experienced nearly ˜5.71- and ˜5.89-fold upregulation in caspase-3 and caspase-9 levels, respectively. Meanwhile, the MM-231 BM cells experienced nearly a ˜2.34 and ˜3.97-fold activation in caspase-3 and caspase-9 levels. Alternatively, MCF-7 BM cells treated with V.M. experienced nearly ˜3.92- and ˜4.15-fold upregulation in caspase-3 and caspase-9 levels, respectively. Meanwhile, the MM-231 BM cells experienced nearly a ˜2.09 and ˜4.31-fold activation in caspase-3 and caspase-9 levels.

### Discussion

The overarching aim of this study was to investigate the biological response to bone metastasized breast cancer when treated with phytochemically enriched plant extracts of O.V. and V.M. Breast cancer cells experience altered growth and chemoresistance when arriving at the bone site. Bone-like ECM formation is observed from osteogenically differentiated MSCs on nano-clay scaffolds with enhanced migration of cancer cells in the presence of bone microenvironment. Two phytochemically-enriched plant extracts, O.V. and V.M., contain many different bioactive phytochemicals. There is a scarcity of non-toxic anti-cancer therapies available, and currently, there is no cure for bone metastasis of breast or prostate cancer.

It is understood that there are more available therapies for hormonal-positive breast cancer compared to triple-negative breast cancer. The IC50 results presented here suggest that O.V. is more suitable for targeting hormonal-positive breast cancer. V.M. is more suitable to target MM231. Furthermore, it was found that treated samples had an increased apoptosis rate compared to non-treated samples, for both O.V. and V.M.

We conducted gene expression experiments to evaluate pro- and anti-apoptotic markers based on these results. The tumor suppressor protein p53 is mainly involved in cell cycle regulation and DNA repair. In response to DNA damage, apoptosis can be triggered by activation of p53. Bcl-2 is part of the anti-apoptotic family of proteins, which are known to be upregulated in chemo-resistant cancer cells. Activation of Bcl-2 proteins leads to inhibition or prevention of cellular apoptosis. We observed activation of p53 in all treated samples. Furthermore, 2D cultures experienced higher fold expression compared to 3D BM cultures. The dysregulation of p53 can be linked to chemoresistance in cancer. Additionally, we found breast cancer cells grown in 3D bone metastatic testbed had higher fold expression of Bcl-2 compared to 2D cultures. Overall, the sequential culture of MSCs with breast cancer cells increased resistance to apoptosis.

Further, we confirmed the initiation of apoptosis by evaluating the expressions of caspases 3 and 9. We observed that ROS levels decreased post-treatment in bone metastatic cultures, while caspase-3 activations were upregulated. Specifically, our results demonstrate that when 3D cultures were treated with all three extracts, the caspase-3 and caspase-9 mRNA expression levels were upregulated. Initiation of apoptosis can be correlated to changes in redox activities. ROS and MtMP were qualitatively measured to understand apoptosis in relation to redox regulation. We observed the mitochondrial membrane potential was lost in the treat bone metastatic cultures. Indicating the plant extracts were promoted apoptosis through reduction in membrane potential, via stress and bioenergetic loss. Overall, our results suggest that phytochemically-enriched O.V., and V.M. can initiate cell death in both primary site and secondary site bone metastatic breast cancer.

The results further show that neither O.V. nor V.M. negatively affected the normal bone tissue. After 24 hours, viability did not change, even with increasing dosages. Lastly, changes in apoptotic expression were not observed in treated bone tissue. Overall, O.V. and V.M. are potential anti-cancer candidates for bone metastatic breast cancer that are also non-toxic to bone.

### Example 5

Patient-derived breast cancer cell lines were utilized to further investigate the effect of the three phytochemically-enriched plant extracts: Rhodiola crenulata (Rhodiola), Vaccinium macrocarpon (cranberry), and Origanum vulgare (oregano). In total, eight different established breast cancer cell lines were developed, details of which are presented in Table 2. The objective of the study was to observed the differences in cell response between the individual patient-derived cell lines.

TABLE 2

| Sample | | Specimen | | Cell line |
|---|---|---|---|---|
| ID | Gender | type | Histology ** | development |
| NT-013 | Female | Tissue | Es, PR receptor-positive, | Cell line |
| | | | Her2-negative | established at |
| | | | | passage-4 |
| NT-015 | Female | Tissue | Es, PR receptor-positive, | Cell line |
| | | | Her2-negative | established at |
| | | | | passage-4 |
| NT-017 | Female | Tissue | Es, PR receptor-positive | Cell line |
| | | | Her2-negative | established at |
| | | | | passage-5 |
| NT-021 | Female | Tissue | Es, PR receptor-negative, | Cell line |
| | | | Her2-positive | established at |
| | | | | passage-3 |
| NT-023 | Female | Tissue | Es, PR receptor-negative, | Cell line |
| | | | Her2-negative | established at |
| | | | | passage-4 |
| NT-042 | Female | Tissue | Es, PR receptor-negative, | Cell line |
| | | | Her2-positive | established at |
| | | | | passage-5 |
| NT-045 | Female | Tissue | Es, PR receptor-negative, | Cell line |
| | | | Her2-negative | established at |
| | | | | passage-7 |
| NT-046 | Female | Tissue | Es, PR receptor-negative, | Cell line |
| | | | Her2-negative | established at |
| | | | | passage-5 |

** Es: Estrogen; PR: Progesterone; Her2: human epidermal growth factor 2

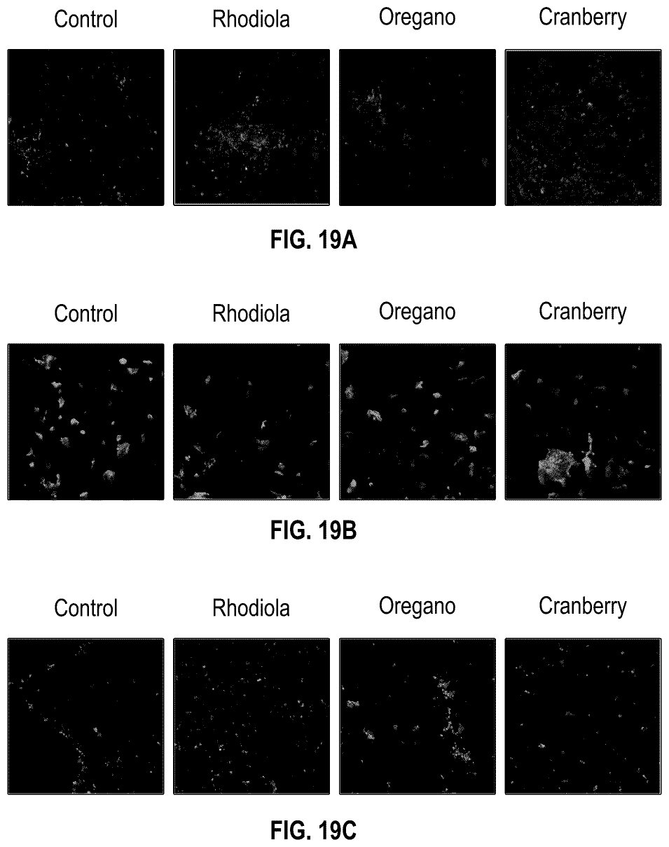

To begin, bone scaffolds were prepared via growth of human mesenchymal stem cells which osteogenically differentiate within 23 days. As a result, bone-extra cellular matrix (ECM) in the scaffolds was obtained. Next, three different patient-derived cell lines (NT023, NT015, NT016) were seeded on the bone-ECM scaffolds and grown for 10-days, creating patient-derived bone-metastatic testbeds. After 10 days, the samples were treated with 800 ppm of Rhodiola, oregano, and cranberry. The 800 ppm dosage was chosen because in Example 4 it was observed that 800 ppm was the highest concentration which was toxic to the cancer cells but completely harmless to the bone cells. FIGS. 19A-C present Live/Dead images of the samples after 24 hours of treatment of each extract. Cell lines NT023 (FIG. 19A) and NT046 (FIG. 19C) had a lot of dead cells, as indicated by the red stain, after 24 hours. NT015 (FIG. 19B) was relatively unaffected by the treatment, with a lot of living cells, indicated by the green stain.

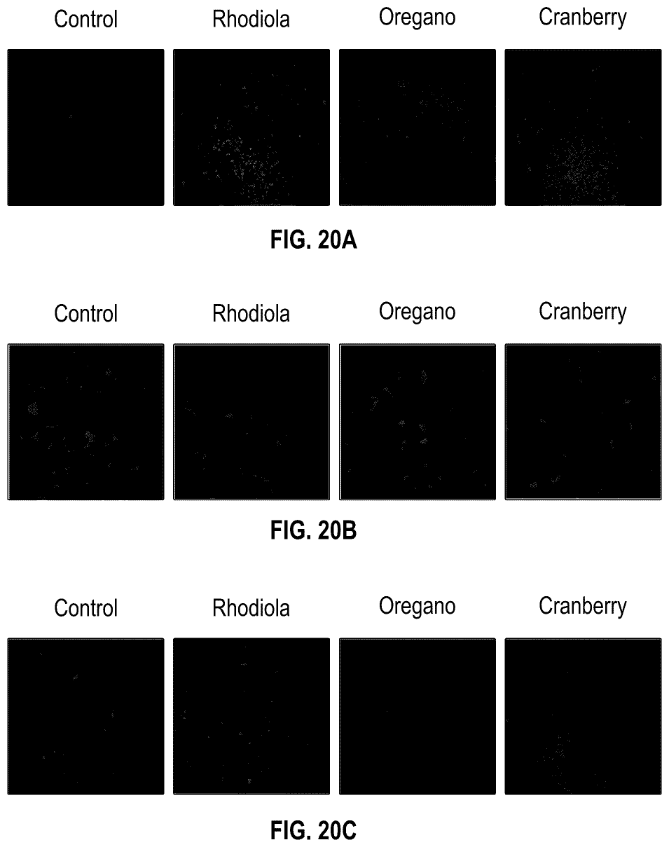

FIGS. 20A-C present immunofluorescent staining of cleaved-caspase 3 (red), a known biomarker for apoptosis activation. Unlike the Live/Dead experiment, this was a 12-hour study. The results are consistent with the Live/Dead images where NT023 (FIG. 20A) and NT046 (FIG. 20C) expressed cleaved-caspase 3 after treatment of all three plant extracts. NT015 (FIG. 20B) was completely unaffected, showing no fluorescent activity.

NT023 and NT046 are both triple-negative breast cancer cell lines. Beneficially, these results support that Rhodiola, oregano, and cranberry extracts have activity against triple-negative breast cancer, which is resistant to typical hormonal therapies.

### Example 6

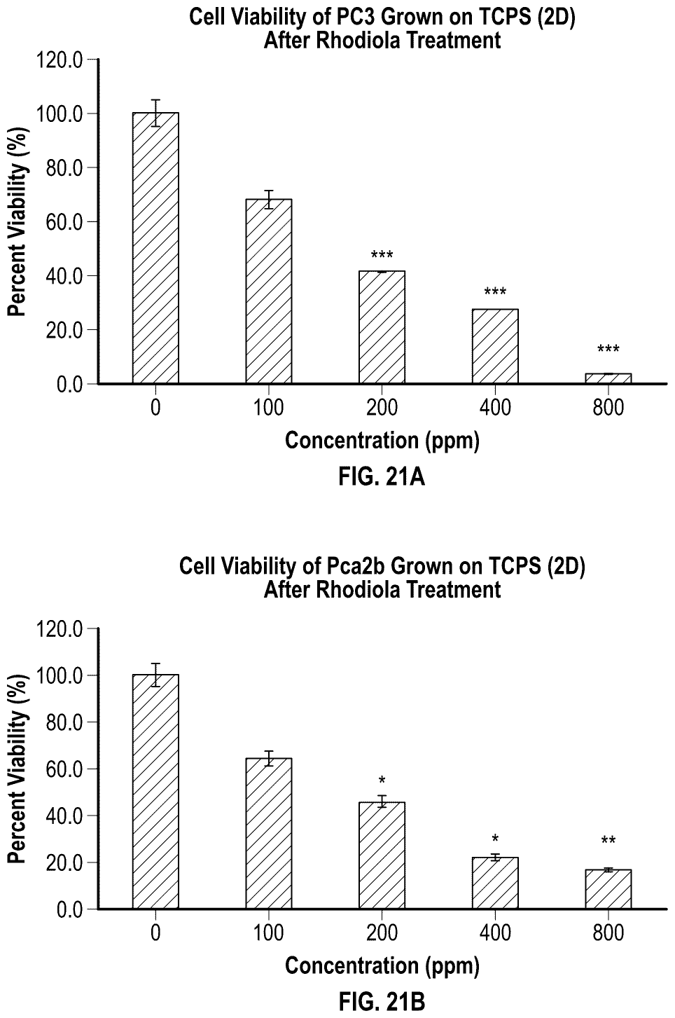

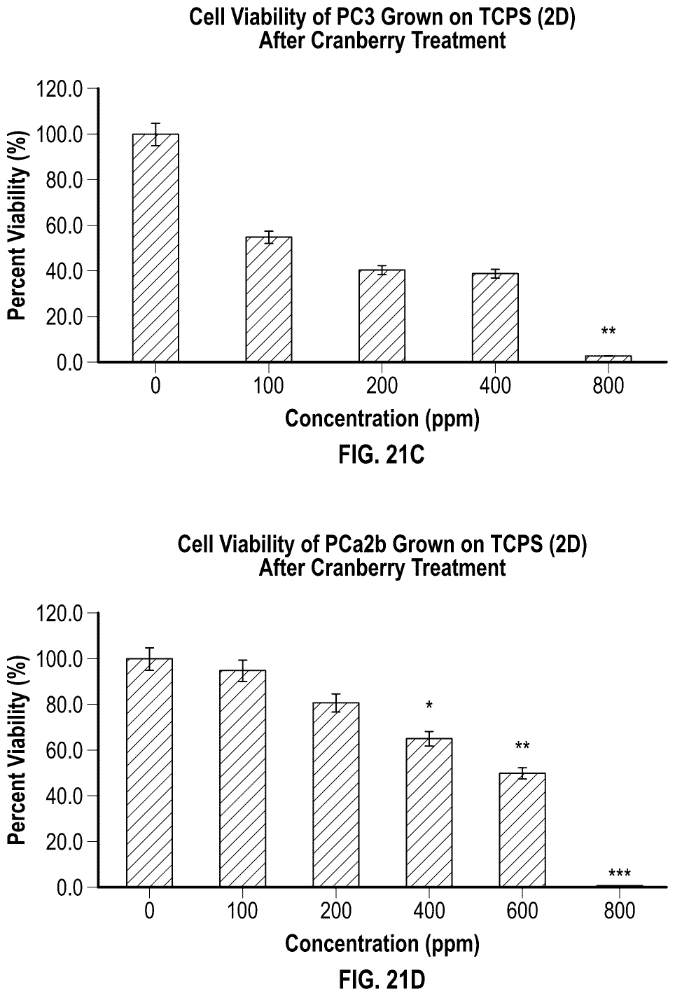

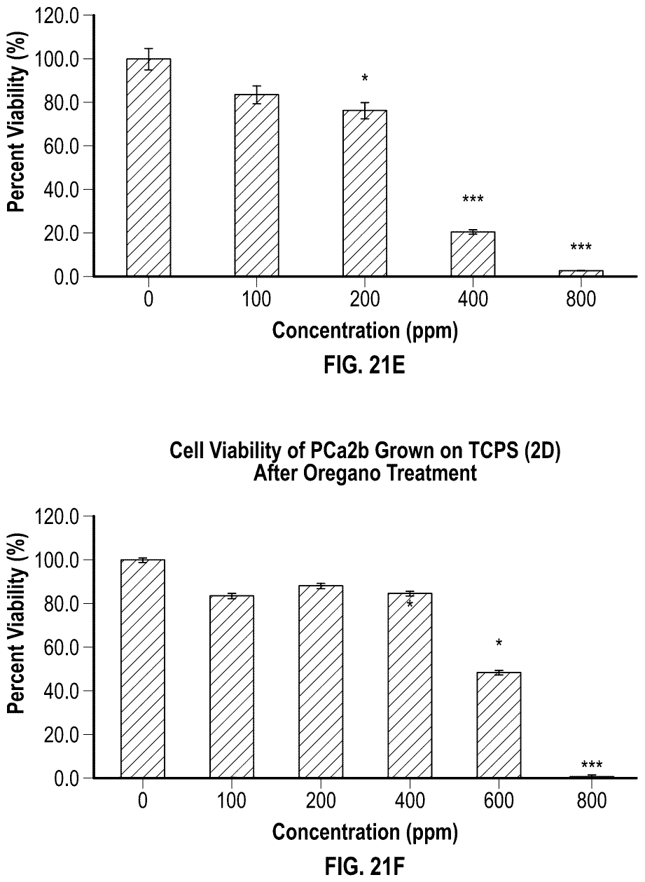

A similar study as in Example 5 was performed using two commercially available prostate cancer cell lines: PC3 and MDA PCa2b. Results are shown in FIGS. 21A-F. FIGS. 21A-B show cell viability of Rhodiola treatment on PC3 and Pca2b, respectively. FIGS. 21C-D show cell viability of cranberry treatment on PC3 and Pca2b, respectively. FIGS. 21E-F show cell viability of oregano treatment on PC3 and Pca2b, respectively.

All three plant extracts were able to significantly reduce the cell viability of both cell lines, cultured in standard monolayer TCPS (tissue cultured polystyrene). Also, as illustrated in the figures, each plant extract-prostate cancer combination required varying dosages or IC50 concentrations (concentration needed to inhibit 50% of activity). The IC50 concentrations of each combination are presented in Table 3.

TABLE 3

| Cell Line | Rhodiola (ppm) | Cranberry (ppm) | Oregano (ppm) |
|---|---|---|---|
| PC3 | 173.1 | 135.3 | 294.2 |
| PCa2b | 179.2 | 601.1 | 590.6 |

Overall, this study further supports that Rhodiola, cranberry, and oregano have anti-cancer activity against prostate cancer cells.

It is to be understood that while the invention has been described in conjunction with the detailed description thereof, the foregoing description is intended to illustrate, and not limit the scope of the invention, which is defined by the scope of the appended claims. Other embodiments, advantages, and modifications are within the scope of the following claims. Any reference to accompanying drawings which form a part hereof, are shown, by way of illustration only. It is understood that other embodiments may be utilized and structural changes may be made without departing from the scope of the present disclosure. All publications discussed and/or referenced herein are incorporated herein in their entirety.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be utilized for realizing the invention in diverse forms thereof.

1. A method for treating a subject with bone metastasis, the method comprising:
administering a composition comprising a phenolic phytochemical of oregano, cranberry, or Rhodiola and an excipient to the subject.

2. The method of claim 1, wherein the bone metastasis is from prostate cancer or breast cancer.

3. The method of claim 1, wherein the bone metastasis is from triple-negative breast cancer.

4. The method of claim 1, wherein the composition comprises an extract of oregano, cranberry, Rhodiola, or a combination thereof.

5. The method of claim 1, wherein the phenolic phytochemical of Rhodiola comprises Gallic acid, 3-O-methylgallic acid, 4-(β-D-Glucopyranosyloxy)-3,5-dimethoxybenzoic acid, protocatechuic acid, vanillic acid, vanillic acid 4-O-β-D-glucopyranoside, Tyrosol, Salidroside, 4-Hydroxybenzoic acid, 4-(beta-D-Glucosyloxy) benzoic acid, Rhodiocyanoside A, Sarmentosin, Epigallocatechin gallate, (7R*,8R*)-3-Methoxy-3′,4,7,9,9′-pentahydroxy-8,4′-oxyneolignan 4-xyloside, Isolariciresinol 4′-O-beta-D-glucoside, Dehydrodiconiferyl alcohol 4-O-beta-D-glucopyranoside, Picein, Icariside D2, Creoside I, Kenposide A, Rhodioloside E, Rhodiooctanoside, coniferoside, dihydroconiferin, triandrin, Vimalin, Pollenitin, Clemastanin A, or a combination thereof.

6. The method of claim 1, wherein the phenolic phytochemical of oregano comprises Carvacrol, Thymol, Creosol, Phytol, P-Cymene, Gamma-Terpinene, 1-Octacosanol, Luteolin 7-O-glucoside, Rosmarinic acid, Luteolin-7-o-glucuronide, Apigenin-7-o-glucuronide, Linalyl acetate, cis-Sabinene hydrate, 4-Hydroxy-4-Methyl-2-Pentanone, Caffeic acid, Trans-Sabinene hydrate, Quercetin 3-O-rutinoside, n-Heptanoic acid, Nitro-L-arginine, Eriodictyol, Taxifolin, Dihydrokaempferol, or a combination thereof.

7. The method of claim 1, wherein the phenolic phytochemical of cranberry comprises Ursolic acid, Cyanidin, Cyanidin 3-O-galactoside, Cyanidin 3-O-arabinoside, Cyanidin 3-O-glucoside, Peonidin, Peonidin 3-O-galactoside, Peonidin 3-O-arabinoside, Peonidin 3-O-glucoside, Malvidin, Malvidin-3-O-galactoside, Malvidin-3-O-arabinoside, Pelargonidin, Pelargonidin 3-O-arabinoside, Delphinidin, Delphinidin-3-O-arabinoside, Petunidin, Petunidin-3-O-galactoside, Quercetin, Hyperin, Avicularin, Quercitrin, Isoquercitrin, Quercetin 3-xyloside, Kaempferol, Kaempferol-3-glucoside, Myricetin, Myricetin 3-alpha-L-arabinofuranoside, -Epicatechin, (+)-Catechin, Epigallocatechin, Epigallocatechin gallate, (−)-Catechin gallate, Gallocatechin gallate, Procyanidin B2, Procyanidin A2, Benzoic acid, Salicylic acid, M-hydroxybenzoic acid, p-Hydroxybenzoic acid, 2,3-Dihydroxybenzoic acid, 2,4-Dihydroxybenzoic acid, 3,4-Dihydroxybenzoic acid, p-hydroxyphenylacetic acid, Vanillic acid, Trans-Cinnamic acid, o-Hydroxycinnamic acid, p-coumaric acid, o-phthalic acid, Ferulic acid, Sinapic acid, Chlorogenic acid, 5-O-Caffeoylquinic acid, Phloridzin, Ellagic acid, cis-Resveratrol, trans-resveratrol, Secoisolariciresinol, Oleanolic acid, Beta-Sitosterol, Sitogluside, Monotropein, Ascorbic acid, Lutein, Niacin, Pantothenic acid, Thiamine, Riboflavin, Adermine, Folic acid, beta-Carotene, alpha-Tocopherol, or a combination thereof.

8. The method of claim 1, wherein the phenolic phytochemical comprises epigallocatechin gallate, Malvidin-3-O-galactoside, Quercitrin, folic acid, procyanidin A2, Sitogluside, or a combination thereof.

9. The method of claim 1, wherein the composition is administered orally.

10. The method of claim 1, wherein the composition is not cytotoxic to healthy bone cells.

11. A composition comprising:
a polymer,
a clay,
a phenolic phytochemical of oregano, cranberry, or Rhodiola,
wherein the polymer and clay form a scaffold, and wherein the phenolic phytochemical coats or is impregnated in the scaffold or is delivered to the scaffold via fluid flow in a bioreactor.

12. The composition of claim 11, wherein the composition comprises an extract of oregano, cranberry, Rhodiola, or a combination thereof.

13. The composition of claim 11, wherein the phenolic phytochemical of Rhodiola comprises Gallic acid, 3-O-methylgallic acid, 4-(β-D-Glucopyranosyloxy)-3,5-dimethoxybenzoic acid, protocatechuic acid, vanillic acid, vanillic acid 4-O-β-D-glucopyranoside, Tyrosol, Salidroside, 4-Hydroxybenzoic acid, 4-(beta-D-Glucosyloxy) benzoic acid, Rhodiocyanoside A, Sarmentosin, Epigallocatechin gallate, (7R*,8R*)-3-Methoxy-3′,4,7,9,9′-pentahydroxy-8,4′-oxyneolignan 4-xyloside, Isolariciresinol 4′-O-beta-D-glucoside, Dehydrodiconiferyl alcohol 4-O-beta-D-glucopyranoside, Picein, Icariside D2, Creoside I, Kenposide A, Rhodioloside E, Rhodiooctanoside, coniferoside, dihydroconiferin, triandrin, Vimalin, Pollenitin, Clemastanin A, or a combination thereof.

14. The composition of claim 11, wherein the phenolic phytochemical of oregano comprises Carvacrol, Thymol, Creosol, Phytol, P-Cymene, Gamma-Terpinene, 1-Octacosanol, Luteolin 7-O-glucoside, Rosmarinic acid, Luteolin-7-o-glucuronide, Apigenin-7-o-glucuronide, Linalyl acetate, cis-Sabinene hydrate, 4-Hydroxy-4-Methyl-2-Pentanone, Caffeic acid, Trans-Sabinene hydrate, Quercetin 3-O-rutinoside, n-Heptanoic acid, Nitro-L-arginine, Eriodictyol, Taxifolin, Dihydrokaempferol, or a combination thereof.

15. The composition of claim 11, wherein the phenolic phytochemical of cranberry comprises Ursolic acid, Cyanidin, Cyanidin 3-O-galactoside, Cyanidin 3-O-arabinoside, Cyanidin 3-O-glucoside, Peonidin, Peonidin 3-O-galactoside, Peonidin 3-O-arabinoside, Peonidin 3-O-glucoside, Malvidin, Malvidin-3-O-galactoside, Malvidin-3-O-arabinoside, Pelargonidin, Pelargonidin 3-O-arabinoside, Delphinidin, Delphinidin-3-O-arabinoside, Petunidin, Petunidin-3-O-galactoside, Quercetin, Hyperin, Avicularin, Quercitrin, Isoquercitrin, Quercetin 3-xyloside, Kaempferol, Kaempferol-3-glucoside, Myricetin, Myricetin 3-alpha-L-arabinofuranoside, -Epicatechin, (+)-Catechin, Epigallocatechin, Epigallocatechin gallate, (−)-Catechin gallate, Gallocatechin gallate, Procyanidin B2, Procyanidin A2, Benzoic acid, Salicylic acid, M-hydroxybenzoic acid, p-Hydroxybenzoic acid, 2,3-Dihydroxybenzoic acid, 2,4-Dihydroxybenzoic acid, 3,4-Dihydroxybenzoic acid, p-hydroxyphenylacetic acid, Vanillic acid, Trans-Cinnamic acid, o-Hydroxycinnamic acid, p-coumaric acid, o-phthalic acid, Ferulic acid, Sinapic acid, Chlorogenic acid, 5-O-Caffeoylquinic acid, Phloridzin, Ellagic acid, cis-Resveratrol, trans-resveratrol, Secoisolariciresinol, Oleanolic acid, Beta-Sitosterol, Sitogluside, Monotropein, Ascorbic acid, Lutein, Niacin, Pantothenic acid, Thiamine, Riboflavin, Adermine, Folic acid, beta-Carotene, alpha-Tocopherol, or a combination thereof.

16. The composition of claim 11, wherein the phenolic phytochemical comprises epigallocatechin gallate, Malvidin-3-O-galactoside, Quercitrin, folic acid, procyanidin A2, Sitogluside, or a combination thereof.

17-18. (canceled)

19. The composition of claim 11, wherein the polymer is a natural polymer, synthetic polymer, or a combination thereof.

20. The composition of claim 11, wherein the polymer comprises albumin, alginate, cellulose, chitin, chitosan, collagen, gelatin, heparin, regenerated silk polymer, polysaccharide, poly(amino acid), polyanhydride, polyester, poly(alpha-hydroxy acid), poly(lactone), poly(orthocarbonate), poly(orthoester), poly(phosphoester), polyphosphazenes, polyacrylonitrile, polycaprolactone, poly(delta-valerolactone), poly(1,5-dioxepan-2-one), poly(epsilon-aprolactone), poly(ester urethane), polygalactouronic acid, poly(gamma-butyrolactone), polyglycolic acid, poly(alpha-hydroxy acids), polyhydroxyalkanoate, polyhydroxybutyric acid, poly(3-hydroxybutyrate-co-3-hydroxyvalerate, polyimide, polylactic acid, poly(lactic-co-glycolic acid), poly(lactic acid-co-caprolactone), poly(trimethylene carbonate), poly-8-valerolactone, chitosan-polygalactouronic acid, polycaprolactone, or a combination thereof.

21-22. (canceled)

23. The composition of claim 11, wherein the clay comprises a smectite, bentonite, beidellite, hectorite, montmorillonite, nontronite, saponite, or combinations thereof.

24-34. (canceled)

35. A method for treating a subject with bone metastasis, the method comprising:
administering the composition of claim 11 to a bone of the subject.

36-47. (canceled)
